(19) United States
(12) Patent Application Publication
(10) Pub. No.: US 2026/0242845 A1
(43) Pub. Date: Aug. 20, 2026

(54) KINASE ACTIVITY EVALUATION APPARATUS AND NON-TRANSITORY COMPUTER READABLE STORAGE MEDIUM

(71) Applicant: CANON MEDICAL SYSTEMS CORPORATION, Otawara-shi (JP)

(72) Inventor: Hiroto SATAKE, Brighton, MA (US)

(73) Assignee: CANON MEDICAL SYSTEMS CORPORATION, Otawara-shi (JP)

(21) Appl. No.: 19/541,983

(22) Filed: Feb. 17, 2026

(30) Foreign Application Priority Data
Feb. 18, 2025 (JP) 2025-024515
Feb. 17, 2026 (JP) 2026-023539

(51) Int. Cl. C12Q 1/48

(52) U.S. Cl. CPC C12Q 1/485

(57) ABSTRACT

An apparatus for evaluating kinase activity according to an embodiment, comprises processing circuitry configured to measure distribution of an immobilization density of a substrate protein and distribution of an amount of phosphorylated amino acid generated in the substrate protein, in a spot containing the substrate protein on a substrate used for measurement; accept a setting related to the distribution of the immobilization density of the substrate protein; and estimate the distribution of the amount of phosphorylated amino acid generated in the substrate protein in the distribution of the immobilization density of the substrate protein determined according to the received setting, based on the distribution of the immobilization density of the substrate protein and the distribution of the amount of phosphorylated amino acid generated in the substrate protein.

### CROSS REFERENCE TO RELATED APPLICATIONS

This application is based upon and claims the benefit of priority from the prior Japanese Patent Applications No. 2025-024515, filed on Feb. 18, 2025, and No. 2026-023539, filed on Feb. 17, 2026, the entire contents of which are incorporated herein by reference.

### FIELD

The embodiments disclosed in this description and the drawings relate to a kinase activity evaluation apparatus and non-transitory computer readable storage medium.

### BACKGROUND

As means for evaluating the activity of kinase contained in a specimen, a technique is known in which the amount of phosphorylated amino acid generated per spot when a spot containing a substrate protein immobilized on a substrate is reacted with the specimen is measured, and the kinase activity is evaluated based on the amount. In this case, the amount of phosphorylated amino acid may be measured based on spot luminance.

In a conventional kinase activity evaluation method, the amount of phosphorylated amino acid of a substrate protein generated per spot is easily affected by the total amount of immobilization of the substrate protein and the distribution of the immobilization density of the substrate protein. In particular, the total amount of immobilization and the density distribution of the substrate protein of each spot on a substrate used in a kinase activity evaluation apparatus are often not set as desired by a user who evaluates kinase activity using the kinase activity evaluation apparatus.

For example, in order to more accurately evaluate the kinase activity, it is desirable to make the total amount of immobilization and the density distribution of substrate proteins the same between spots containing various substrate proteins on the substrate used in the kinase activity evaluation apparatus, or to make the concentration of the immobilized substrate protein in the spot the same as that of a specimen or make the amount ratio of each substrate protein in the spot the same as an expression ratio in the specimen in order to reproduce the same (or close) environment as that of the specimen in the spot containing the substrate protein on the substrate. However, there is a problem that it is difficult to realize such a substrate since a large amount of cost and labor are required to produce the substrate.

### BRIEF DESCRIPTION OF THE DRAWINGS

FIG. 1 is a block diagram illustrating an example of the mechanism configuration of a kinase activity evaluation apparatus according to a first embodiment.

FIG. 2 is a diagram illustrating an example of the flow of kinase activity evaluation performed using the kinase activity evaluation apparatus according to the first embodiment.

FIG. 3 is a diagram illustrating an example of a method for measuring the distribution of the amount of phosphorylated amino acid generated in a substrate protein and the distribution of a substrate protein immobilization density.

FIG. 4 is a diagram illustrating an example of a measurement result in the first embodiment.

FIG. 5 is a diagram illustrating an example of a relationship between the distribution of the amount of phosphorylated amino acid for each substrate protein and the distribution of the immobilization density of the substrate protein according to the first embodiment.

FIG. 6 is a diagram illustrating an example of a relationship between the distribution of the amount of phosphorylated amino acid for each substrate protein and the distribution of the immobilization density of the substrate protein according to the first embodiment.

FIG. 7 is a diagram illustrating an example of a substrate protein expression level in a specimen.

FIG. 8 is a diagram illustrating an example of a template for setting a parameter.

FIG. 9 is a diagram illustrating an example of a parameter setting result according to the first embodiment.

FIG. 10 is a diagram illustrating an example of the setting of the distribution of a substrate protein immobilization density in the first embodiment.

FIG. 11 is a diagram illustrating an example of an estimation result in the first embodiment.

FIG. 12 is a diagram illustrating an example of a method for outputting an estimation result in the first embodiment.

FIG. 13 is a diagram illustrating an example of the flow of kinase activity evaluation performed using a kinase activity evaluation apparatus according to a first modification.

FIG. 14 is a diagram illustrating an example of the flow of kinase activity evaluation performed using a kinase activity evaluation apparatus according to a second modification.

FIG. 15 is a diagram illustrating an example of a parameter setting result in the second modification.

FIG. 16 is a diagram illustrating an example of the setting of the distribution of a substrate protein immobilization density in the second modification.

FIG. 17 is a diagram illustrating another example of a parameter setting result in the second modification.

FIG. 18 is a diagram illustrating another example of the setting of the distribution of the substrate protein immobilization density in the second modification.

FIG. 19 is a diagram illustrating an example of a relationship between the distribution of the amount of phosphorylated amino acid for each substrate protein and the distribution of the immobilization density of the substrate protein according to a third modification.

FIG. 20 is a diagram illustrating an example of an estimation result in the third modification.

FIG. 21 is a diagram illustrating an example of a method for outputting the estimation result in the third modification.

FIG. 22 is a diagram illustrating another example of the relationship between the distribution of the amount of phosphorylated amino acid for each substrate protein and the distribution of the immobilization density of the substrate protein according to the third modification.

FIG. 23 is a diagram illustrating an example of the setting of the distribution of a substrate protein immobilization density in a fourth modification.

FIG. 24 is a diagram illustrating an example of a method for outputting an estimation result in the fourth modification.

FIG. 25 is a diagram illustrating an example of the setting of the distribution of a substrate protein immobilization density in the fourth modification.

FIG. 26 is a diagram illustrating an example of the distribution of the immobilization density of MERTK at each point within a spot corresponding to each pixel and the distribution of the amount of phosphorylated tyrosine generated in MERTK.

FIG. 27 is a diagram illustrating an example of the correspondence relationship of the amount of phosphorylated tyrosine with respect to the immobilization density of MERTK.

FIG. 28 is a diagram illustrating an example of a parameter setting result.

FIG. 29 is a diagram illustrating an example of the amount of phosphorylated amino acid generated in a substrate protein in the distribution of the immobilization density of the substrate protein set in a desired manner.

### DETAILED DESCRIPTION

Hereinafter, embodiments of a kinase activity evaluation apparatus and non-transitory computer readable storage medium will be described in detail with reference to the drawings.

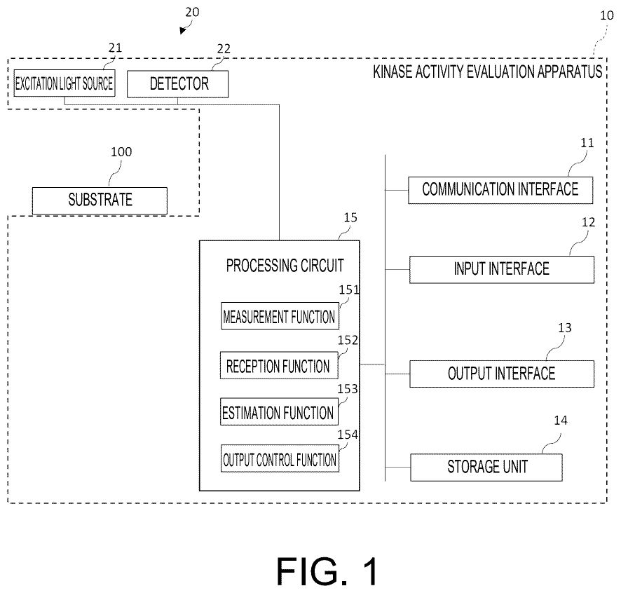

FIG. 1 is a block diagram illustrating an example of the mechanism configuration of a kinase activity evaluation apparatus according to a first embodiment. A kinase activity evaluation apparatus 10 according to the first embodiment is an apparatus for evaluating kinase activity. Specifically, the kinase activity evaluation apparatus 10 is an apparatus for evaluating kinase activity, the apparatus measures distribution of an immobilization density of a substrate protein and distribution of an amount of phosphorylated amino acid generated in the substrate protein, in a spot containing the substrate protein on a substrate used for measurement, accepts a setting related to the distribution of the immobilization density of the substrate protein; and estimates the distribution of the amount of phosphorylated amino acid generated in the substrate protein in the distribution of the immobilization density of the substrate protein determined according to the received setting, based on the distribution of the immobilization density of the substrate protein and the distribution of the amount of phosphorylated amino acid generated in the substrate protein.

The “substrate” in the kinase activity evaluation apparatus 10 according to the first embodiment means a carrier for immobilizing a substrate protein, and is not particularly limited, and examples thereof include a two-dimensional plate, a polymer gel, a fiber and a fiber sheet, a bead, and a rod. The surface of the “substrate” may be smooth, but may have a porous structure or a micro/nanostructure such as a fiber. The “substrate” is preferably a two-dimensional plate, and the two-dimensional plate includes a plate substrate such as a slide glass or a cover glass, a well substrate such as an array plate, or the like. The “substrate” is more preferably a well substrate such as an array plate. The array plate has a spot containing a substrate protein, is used for the comprehensive analysis of a sample, and is also called a microchip, a microarray, a protein chip, or the like.

The “substrate protein” in the kinase activity evaluation apparatus 10 according to the first embodiment is not particularly limited as long as the substrate protein satisfies both of the fact that the substrate protein can be physically or chemically immobilized on the substrate and the fact that means capable of measuring the amount of the “substrate protein” is provided, and specific examples thereof include proteins and peptides. The “substrate protein” may be immobilized on the substrate in such a manner that the substrate protein is directly bound to the substrate, may be bound to the substrate via a linker substance, or may be immobilized in such a manner that the substrate protein is contained in a gel substance.

The “spot” in the kinase activity evaluation apparatus 10 according to the first embodiment means a certain closed region in which the substrate protein is immobilized on the substrate. The form of the “spot” is not particularly limited, and may be, for example, a square, a rectangle, a circle, an ellipse, or the like, but is preferably a circle. The “spot” may be continuous or discontinuous in a two-dimensional plane. In the “spot”, the substrate protein may be immobilized two-dimensionally or three-dimensionally depending on the substrate. The substrate protein to be immobilized on the “spot” may be one kind, or two or more kinds of substrate proteins may be immobilized on the same region when the amount of phosphorylated amino acid generated in two or more kinds of substrate proteins and the amount of phosphorylated amino acid generated in each of the two or more kinds of substrate proteins can be identified and measured per kind.

Herein, one closed region in which the density of the immobilized substrate protein is uniform is defined as a “compartment”. Here, the expression that the density of the immobilized substrate protein is uniform means that it is sufficient that the density is substantially uniform, and does not mean that it is not permitted to include a region having a non-uniform density. The “compartment” may have the same or different density of the immobilized substrate protein with respect to other “compartments”. The “compartment” may be continuous or discontinuous in a two-dimensional plane with respect to other “compartments”. The form of the “compartment” is not particularly limited, and may be, for example, a square, a rectangle, a circle, an ellipse, or the like. The uniform density of the immobilized substrate protein may be an average density in one closed region, and thus the density of the immobilized substrate protein in the compartment may be the average density of the immobilized substrate protein in the same region.

The compartment may be selected with a spot as one unit, or may be selected from one closed region having a uniform density of the immobilized substrate protein that is smaller than one spot and can be plurally set within one spot. Here, the expression that the density of the immobilized substrate protein is uniform means that it is sufficient that the density is substantially uniform, and does not mean that it is not permitted to include a region having a non-uniform density. Such one closed region is referred to as a “virtual compartment”, and the “virtual compartment” is one unit of the compartment. The size and form of the “virtual compartment” may be arbitrarily determined. Similarly to the “compartment”, the “virtual compartment” may have the same or different density of the immobilized substrate protein with respect to other “virtual compartments”. The “virtual compartment” may be continuous or discontinuous in the two-dimensional plane with respect to other “virtual compartments”. The form of the “virtual compartment” is not particularly limited, and may be, for example, a square, a rectangle, a circle, an ellipse, or the like. Examples of the “virtual compartment” include a compartment in which a spot having a size of several thousand μm² including a region in which the density of the immobilized substrate protein is different is set to a square form of about 1 pixel (10 μm×10 μm). The uniform density of the immobilized substrate protein may be an average density in one closed region, and thus the density of the immobilized substrate protein in the “virtual compartment” may be the average density of the immobilized substrate protein in the same region.

As illustrated in FIG. 1, the kinase activity evaluation apparatus 10 includes a communication interface 11, an input interface 12, an output interface 13, a storage unit 14, a processing circuit 15, and an optical system 20.

The communication interface 11 implements various communication protocols according to the form of a network. The communication interface 11 realizes communication with other devices via a network according to various communication protocols.

The input interface 12 is a circuit that receives various inputs from a user. The input interface 12 is realized by, for example, a mouse, a keyboard, a trackball, a manual switch, a foot switch, a button, a joystick, or the like.

The output interface 13 outputs various images and information. For example, the output interface 13 outputs the estimation result of the distribution of the amount of phosphorylated amino acid by an estimation function 153 described later, a graphical user interface (GUI) for receiving various operations from the user, and the like. The output interface 13 includes, for example, a liquid crystal display, a cathode ray tube (CRT) display, a speaker, and the like. The output interface 13 corresponds to an output unit in the present embodiment.

The storage unit 14 is realized by, for example, a random access memory (RAM), a semiconductor memory element such as a flash memory, a hard disk, an optical disk, or the like.

The processing circuit 15 is an arithmetic circuit that performs various operations. The processing circuit 15 includes, for example, a processor. The processing circuit 15 according to the present embodiment estimates, for example, the distribution of the amount of phosphorylated amino acid generated in the substrate protein in the distribution of the immobilization density of the substrate protein determined according to a setting, thereby evaluating the kinase activity.

As illustrated in FIG. 1, the processing circuit 15 includes a measurement function 151, a reception function 152, an estimation function 153, and an output control function 154. The measurement function 151 corresponds to a measurement unit in the present embodiment, the reception function 152 corresponds to a reception unit in the present embodiment, the estimation function 153 corresponds to an estimation unit in the present embodiment, and the output control function 154 corresponds to an output control unit in the present embodiment.

In the embodiment illustrated in FIG. 1, in the processing circuit 15, processing functions performed by the measurement function 151, the reception function 152, the estimation function 153, and the output control function 154 are stored in the storage unit 14 in the form of a program executable by a computer. The processing circuit 15 is a processor that realizes a function corresponding to each program by reading and executing the program from the storage unit 14. In other words, the processing circuit 15 in a state of reading each program has each function illustrated in the processing circuit 15 of FIG. 1. Note that, in FIG. 1, it has been described that the measurement function 151, the reception function 152, the estimation function 153, and the output control function 154 are realized in the single processing circuit 15, but these functions may be realized by combining a plurality of independent processors to constitute the processing circuit 15 and executing a program by each processor.

The measurement function 151 is a function of measuring the distribution of the immobilization density of the substrate protein and the distribution of the amount of phosphorylated amino acid generated in the substrate protein, in a spot containing the substrate protein on a substrate 100 used for measurement. Specifically, the measurement function 151 measures the distribution of the immobilization density for each type of substrate protein and the distribution of the amount of phosphorylated amino acid generated for each type of substrate protein, in a spot containing one or more types of substrate proteins on the substrate used for measurement. In the measurement function 151, for example, the distribution of the immobilization density of the substrate protein and the distribution of the amount of phosphorylated amino acid generated in the substrate protein are measured using an optical system 20. The measurement function 151 may further have a function of acquiring a relationship between the distribution of the immobilization density of the substrate protein measured in the measurement function 151 and the distribution of the amount of phosphorylated amino acid generated in the substrate protein. The measurement function 151 may be a function of acquiring a relationship between the distribution of the immobilization density for each type of substrate protein measured in the measurement function 151 and the distribution of the amount of phosphorylated amino acid generated for each type of substrate protein.

In the kinase activity evaluation apparatus 10 according to the first embodiment, the measurement function 151 acquires the relationship between the distribution of the immobilization density of the substrate protein measured in the measurement function 151 and the distribution of the amount of phosphorylated amino acid generated in the substrate protein, but the relationship between the distribution of the immobilization density of the substrate protein and the distribution of the amount of phosphorylated amino acid generated in the substrate protein may be acquired other than by the measurement function 151. For example, the processing circuit 15 of the kinase activity evaluation apparatus 10 according to the first embodiment may have an acquisition function of acquiring the relationship between the distribution of the immobilization density of the substrate protein measured in the measurement function 151 and the distribution of the amount of phosphorylated amino acid generated in the substrate protein.

In the kinase activity evaluation apparatus 10 according to the first embodiment, the measurement function 151 acquires the relationship between the distribution of the immobilization density for each type of substrate protein measured in the measurement function 151 and the distribution of the amount of phosphorylated amino acid generated for each type of substrate protein, but the relationship between the distribution of the immobilization density for each type of substrate protein and the distribution of the amount of phosphorylated amino acid generated for each type of substrate protein may be acquired other than by the measurement function 151. For example, the kinase activity evaluation apparatus 10 according to the first embodiment may have an acquisition function of acquiring the relationship between the distribution of the immobilization density for each type of substrate protein measured in the measurement function 151 and the distribution of the amount of phosphorylated amino acid generated for each type of substrate protein.

The measurement function 151 measures the distribution of the immobilization density of the substrate protein and the distribution of the amount of phosphorylated amino acid generated in the substrate protein using the optical system 20. However, a method in which the measurement function 151 measures the distribution of the immobilization density of the substrate protein and the distribution of the amount of phosphorylated amino acid generated in the substrate protein is not limited to the case of using the optical system 20. That is, the method in which the measurement function 151 measures the distribution of the immobilization density of the substrate protein and the distribution of the amount of phosphorylated amino acid generated in the substrate protein is arbitrary.

In the kinase activity evaluation apparatus 10 according to the first embodiment, the density of the immobilized substrate protein may be acquired by measuring the amount of the substrate protein in a certain region and dividing it by the area of the same region, or when the value is known, the value may be acquired. The amount of the substrate protein in a certain region can be measured by a known method. Examples thereof include quantification by a surface plasmon resonance (SPR) method, quantification based on electrochemical characteristics (for example, potential, current value, impedance, capacitance, and the like), quantification based on the presence distribution of elements obtained by X-ray spectroscopy or the like, quantification using an AFM, quantification based on signal information derived from a labeling substance introduced into the substrate protein, and the like, and quantification is preferably based on signal information derived from a labeling substance introduced into the substrate protein. The labeling substance is not particularly limited, but is preferably optically detectable. Examples of such a labeling substance include a fluorescent substance, a light-absorbing substance, a light-emitting substance, a scattering substance, a polarizing substance, and an oxidation-reduction substance.

In the kinase activity evaluation apparatus 10 according to the first embodiment, the amount of phosphorylated amino acid generated in the substrate protein can be measured by, for example, generating phosphorylated amino acid in the substrate protein and measuring the amount of phosphorylated amino acid generated. The amount of phosphorylated amino acid generated may be measured by direct quantification, or may be measured by indirect quantification, for example, by further labeling the phosphorylated substrate protein to generate labeled phosphorylated amino acid in the phosphorylated substrate protein, and performing quantification based on signal information derived from the labeling substance.

Further labeling the phosphorylated substrate protein means introducing the labeling substance into the phosphorylated substrate protein via a phosphorylation site recognition substance. The phosphorylation site recognition substance refers to a substance that specifically recognizes a phosphorylated site in a substrate protein that has been phosphorylated by kinase. The phosphorylation site recognition substance is not particularly limited, and examples thereof include an anti-phosphorylated amino acid antibody. When the labeling substance is not bound to the phosphorylation site recognition substance, the labeling substance may be introduced through a specific reaction including a secondary antibody or a biotin avidin reaction.

Examples of the labeling substance include a radioactive substance, an enzyme, a capture molecule, a fluorescent substance, a light-emitting substance, and metal particles, and the labeling substance is preferably optically detectable. Examples of such a labeling substance include a fluorescent substance, a chemiluminescent substance, a phosphorescent substance, a dye, gold nanoparticles, fluorescent particles, an enzyme for enzyme chemiluminescence or color reaction, and fine particles having absorption at a specific wavelength. The labeling substance may contain an antibody, a ligand, or another binding site. The binding or introduction of the labeling substance includes binding of the labeling substance by a hydrophobic interaction, an electrostatic interaction, a van der Waals interaction, a hydrogen bond, or a covalent bond, introduction at the time of synthesis, and other known methods as labeling methods.

The amount of phosphorylated amino acid generated in the labeled phosphorylated substrate protein is quantified based on signal information derived from the labeling substance. The intensity of the signal provides information on the amount of the labeled phosphorylated substrate protein, i.e. makes it possible to quantify the amount of phosphorylated amino acid generated in the labeled phosphorylated substrate protein. Such a signal is preferably optically detectable, and examples thereof include light intensity information from a spot, and spectral information.

When the labeling substance is a fluorescent substance, the optical system 20 can be used without limitation as long as the fluorescent substance is excited and fluorescence can be detected. An excitation light source 21 can be used to excite the fluorescent substance, and examples of such an excitation light source 21 include a laser light source, a light emitting diode, an LED, a mercury arc lamp, and a tungsten halogen lamp. For detection, a detector 22 such as a CCD camera or a photodiode may be used. The optical system 20 appropriately includes a filter and emits or detects light having a limited wavelength. The optical system 20 may include a lens. The optical system 20 may be a scanning type or a non-scanning type. Specifically, a confocal optical unit can be used as the optical system 20.

When chemiluminescence, a dye, or the like is used as the labeling substance, the excitation light source may not be provided.

In the kinase activity evaluation apparatus 10 according to the first embodiment, a method for measuring the distribution of the immobilization density of a substrate protein and the distribution of the amount of phosphorylated amino acid generated in the substrate protein, and acquiring the relationship between the measured distribution of the immobilization density of the substrate protein and the distribution of the amount of phosphorylated amino acid generated in the substrate protein is not particularly limited, but may be performed based on the variation in the immobilization density of the substrate protein formed within one spot. Examples of such a method include a method for obtaining the amount of the immobilized substrate protein of each pixel within a spot and the amount of phosphorylated amino acid generated in the same substrate protein from a fluorescence image illustrating the amount of the immobilized substrate protein within one spot and the amount of phosphorylated amino acid generated in the same substrate protein to create two-dimensional distribution.

In the kinase activity evaluation apparatus 10 according to the first embodiment, a method for measuring the distribution of the immobilization density for each type of substrate protein and the distribution of the amount of phosphorylated amino acid generated for each type of substrate protein, and acquiring the relationship between the measured distribution of the immobilization density for each type of substrate protein and the distribution of the amount of phosphorylated amino acid generated for each type of substrate protein is not particularly limited, but may be performed based on the variation in the immobilization density of the substrate protein formed within one spot. Examples of such a method include a method for obtaining the amount of the immobilized substrate protein in each pixel within the spot for each type of substrate protein and the amount of phosphorylated amino acid generated in the same substrate protein from a fluorescence image illustrating the amount of the immobilized substrate protein within one spot and the amount of phosphorylated amino acid generated in the same substrate protein to create two-dimensional distribution.

The method for producing the variation in the immobilization density of the substrate protein in each pixel within one spot is not particularly limited, but for example, a plurality of spots (for example, a spot series prepared using a substrate protein solution in which the substrate protein concentration is stepwisely increased to 1 time, 2 times, 4 times, 8 times, or the like) prepared in advance so that the immobilization density of the substrate protein is different may be used to produce the variation.

Returning to FIG. 1, the reception function 152 is a function of receiving a setting related to the distribution of the immobilization density of the substrate protein. Specifically, the reception function 152 is a function of accepting a setting related to the distribution of the immobilization density for each type of substrate protein. The reception function 152 may accept a setting related to the distribution of the immobilization density of the substrate protein acquired or stored in the kinase activity evaluation apparatus 10 according to the first embodiment, or may accept a setting related to the distribution of the immobilization density of the substrate protein input by the user of the apparatus. Alternatively, the reception function 152 may receive a setting related to the distribution of the immobilization density of the substrate protein acquired via a network. The reception function 152 may accept a setting related to the distribution of the immobilization density for each type of substrate protein acquired or stored in the kinase activity evaluation apparatus according to the first embodiment, may accept a setting related to the distribution of the immobilization density for each type of substrate protein set by the user of the apparatus, or may accept a setting related to the distribution of the immobilization density for each type of substrate protein acquired via a network.

Note that the setting related to the distribution of the immobilization density of the substrate protein accepted by the reception function 152 of the kinase activity evaluation apparatus 10 according to the first embodiment is not particularly limited, and the target of the setting may be a spot or may not be a spot as long as it is a region containing the substrate protein.

The setting related to the distribution of the immobilization density for each type of substrate protein accepted by the reception function 152 of the kinase activity evaluation apparatus 10 according to the first embodiment is not particularly limited, and the target of the setting may be a spot or may not be a spot as long as it is a region containing the substrate protein.

When the target of setting is the spot, the setting related to the distribution of the immobilization density of the substrate protein or the setting related to the distribution of the immobilization density for each type of substrate protein is not particularly limited as long as it is the setting of a parameter related to the spot, but preferably includes one or more settings selected from the group consisting of the area and shape of each spot and the total amount of the immobilized substrate protein in each spot, and more preferably includes the setting of the total amount of the immobilized substrate protein in each spot. The number of parameters related to these spots may be one or two or more. The parameters related to these spots may be arbitrarily determined for each spot, and may be the same or different between the spots. Furthermore, the parameters related to these spots may be the same or different between different types of substrate proteins, but preferably the areas and shapes of the spots are the same. In addition, when there is a preset template, the parameters related to these spots may be determined by selecting the template.

When one of the parameters related to the spots is the total amount of the immobilized substrate protein in each spot, preferably, the ratio of the amount of another specific substrate protein to the amount of a specific substrate protein in each spot and the ratio of the amount of another specific substrate protein to the amount of a specific substrate protein in the specimen containing the kinase to be evaluated are at least partially the same, and more preferably all the same. In this case, it is more preferable that the areas of the spots are the same.

The area of the spot and the total amount of the immobilized substrate protein in the spot, which are parameters related to the spot, are preferably determined such that the concentration of the substrate protein within the spot (concentration in consideration of the spot thickness) is equal to or not lower than the concentration of the substrate protein in the specimen. For example, when the spot thickness is 5 nm (size of a typical spherical protein), it is preferable that the concentration of the substrate protein within the spot obtained by the following formula:

Σ substrate protein immobilization density (g/nm²)/spot thickness (5 nm) [Mathematical formula 1]
is set to be equal to or higher than the concentration in the specimen.

In the parameter setting of the spot, the total amount of immobilization and the immobilization density distribution may be different among the substrate protein species, but in order to make the amount of kinase supplied to the spot uniform, it is preferable to make the spot shape and the area constant among the substrate protein species. When the distribution range of the immobilization density is simply set, the distribution range of the immobilization density may be the same or different for each substrate protein.

The “specimen” in the kinase activity evaluation apparatus 10 according to the first embodiment can be appropriately determined according to the purpose, and anything including kinase can be the “specimen”. The “specimen” includes a biologically derived substance, an extract from a living body or the like, blood, a blood-derived substance, a food, a food-derived substance, a natural product, a natural product-derived substance, a culture solution-derived substance, and the like. The “specimen” may be appropriately pretreated according to the purpose or procedure, or a reagent may be added in advance. The form of the “specimen” may be a gas, a solid, or a liquid, but the specimen is used in a liquid form through dilution, suspension, or extraction using water, physiological saline, a buffer solution, or other solutions as appropriate. The specimen may contain an antiseptic agent or other additives. A reagent is added to the “specimen” according to the purpose.

The total amount of the immobilized substrate protein in the spot, which is a parameter related to the spot, is preferably determined based on the substrate protein in the specimen containing the kinase to be evaluated, but when there is data related to a sample similar to the specimen, it may be determined based on the data. Examples of such a similar sample include a model cell line of a disease that is the same as or similar to a disease affecting a patient from which the specimen was derived, and a living body-derived tissue.

When the target to be set is not the spot, the setting related to the distribution of the immobilization density of the substrate protein may include, for example, the setting of the distribution range of the immobilization density of the substrate protein. The distribution range of the immobilization density of the substrate protein may be the same as or different from the distribution range of the immobilization density of the substrate protein present on the substrate actually used for measurement in the kinase activity evaluation apparatus.

When the target to be set is not the spot, the setting related to the distribution of the immobilization density for each type of substrate protein may include, for example, the setting of the distribution range of the immobilization density of the substrate protein. When the distribution range of the immobilization density of the substrate protein is set, the distribution range of the immobilization density may be the same or different for each type of substrate protein. The distribution range of the immobilization density of the substrate protein may be the same as or different from the distribution range of the immobilization density of the substrate protein present on the substrate actually used for measurement in the kinase activity evaluation apparatus.

Returning to FIG. 1, the estimation function 153 is a function of estimating the distribution of the amount of phosphorylated amino acid generated in the substrate protein in the distribution of the immobilization density of the substrate protein determined according to the received setting based on the distribution of the immobilization density of the substrate protein and the distribution of the amount of phosphorylated amino acid generated in the substrate protein. Specifically, the estimation function 153 estimates the distribution of the amount of phosphorylated amino acid generated for each type of substrate protein in the distribution of the immobilization density for each type of substrate protein determined according to the received setting based on the distribution of the immobilization density for each type of substrate protein and the distribution of the amount of phosphorylated amino acid generated for each type of substrate protein.

In the estimation function 153 of the kinase activity evaluation apparatus 10 according to the first embodiment, the relationship between the measured distribution of the immobilization density of the substrate protein and the distribution of the amount of phosphorylated amino acid generated in the substrate protein may be determined by fitting the two, that is, based on fitting. For example, it is possible to estimate the amount of phosphorylated amino acid generated in the substrate protein by extrapolating a fitting curve for a range exceeding the range of the immobilization density of the substrate protein present on the substrate actually used for measurement in the kinase activity evaluation apparatus.

In the estimation function 153 of the kinase activity evaluation apparatus 10 according to the first embodiment, the relationship between the measured distribution of the immobilization density for each type of substrate protein and the distribution of the amount of phosphorylated amino acid generated for each type of substrate protein may be determined by fitting the two, that is, based on fitting. For example, it is possible to estimate the amount of phosphorylated amino acid generated in the substrate protein by extrapolating a fitting curve for a range exceeding the range of the immobilization density of the substrate protein present on the substrate actually used for measurement in the kinase activity evaluation apparatus.

In the estimation function 153 of the kinase activity evaluation apparatus 10 according to the first embodiment, the value of the amount of phosphorylated amino acid generated in the substrate protein with respect to the measured value of the immobilization density of the substrate protein may be expressed as a constant value, or may be expressed as probability distribution. Therefore, in the kinase activity evaluation apparatus 10 according to the first embodiment, the relationship between the measured distribution of the immobilization density of the substrate protein and the distribution of the amount of phosphorylated amino acid generated in the substrate protein may be determined based on the probability distribution, and the relationship between the measured distribution of the immobilization density for each type of substrate protein and the distribution of the amount of phosphorylated amino acid generated for each type of substrate protein may be determined based on the probability distribution. In this case, the probability distribution of the amount of phosphorylated amino acid generated in the substrate protein with respect to the measured value of the immobilization density of the substrate protein may be expressed in a form having normal distribution. This probability distribution can be determined from the distribution of the amount of phosphorylated amino acid generated in the same substrate protein with respect to the value of the immobilization density of the substrate protein present on the substrate actually used for measurement in the kinase activity evaluation apparatus 10. For example, one spot may be divided into a plurality of “virtual compartments” that are smaller than one spot and can be set within one spot, and probability distribution based on normal distribution may be acquired by determining the average value and standard deviation of the amount (this amount may be, for example, an immobilization density) of phosphorylated amino acid generated in the immobilized substrate protein in each “virtual compartment”. For example, when the amount of phosphorylated amino acid generated in the substrate protein is estimated by extrapolating a fitting curve for a range exceeding the range of the immobilization density of the substrate protein actually present on the substrate used for measurement in the kinase activity evaluation apparatus 10, the probability distribution of the region expanded by the extrapolation can be estimated based on the probability distribution of the amount of phosphorylated amino acid generated in the substrate protein with respect to the value of the immobilization density of the substrate protein actually present on the substrate actually used for measurement in the kinase activity evaluation apparatus 10.

A method in which the estimation function 153 estimates the distribution of the amount of phosphorylated amino acid generated in the substrate protein in the distribution of the immobilization density of the substrate protein determined according to the received setting based on the distribution of the immobilization density of the substrate protein and the distribution of the amount of phosphorylated amino acid generated in the substrate protein is not particularly limited. For the distribution of the immobilization density of the substrate protein and the distribution of the amount of phosphorylated amino acid generated in the substrate protein, the result measured by the measurement function 151 may be acquired and used as it is, or the stored measurement result may be acquired and used.

A method in which the estimation function 153 estimates the distribution of the amount of phosphorylated amino acid generated for each type of substrate protein in the distribution of the immobilization density for each type of substrate protein determined according to the received setting based on the distribution of the immobilization density for each type of substrate protein and the distribution of the amount of phosphorylated amino acid generated for each type of substrate protein is not particularly limited. For the distribution of the immobilization density for each type of substrate protein and the distribution of the amount of phosphorylated amino acid generated for each type of substrate protein, the result measured by the measurement function 151 may be acquired and used as it is, or the stored measurement result may be acquired and used.

When the value of the amount of phosphorylated amino acid generated in the substrate protein with respect to the measured value of the immobilization density of the substrate protein is expressed as a constant value, the estimation function 153 can estimate, for example, the value of the amount of phosphorylated amino acid at each pixel within one spot determined according to the distribution of the desired substrate protein immobilization density based on a fitting curve.

When the value of the amount of phosphorylated amino acid generated in the substrate protein with respect to the measured value of the immobilization density of the substrate protein is expressed as probability distribution, the estimation function 153 can perform the estimation by, for example, estimating the value of the amount of phosphorylated amino acid at each pixel within one spot determined according to the distribution of the desired substrate protein immobilization density using a random number based on the probability distribution, or estimating the value of the amount of phosphorylated amino acid at each pixel within one spot determined according to the distribution of the desired substrate protein immobilization density as the probability distribution.

The kinase activity evaluation apparatus 10 according to the first embodiment may have a function of evaluating the kinase activity based on the value of the amount of phosphorylated amino acid generated in the substrate protein with respect to the value of the immobilization density of the substrate protein estimated by the estimation function 153. This evaluation includes assuming that the estimated result is the activity of the kinase to be evaluated.

The output control function 154 controls the output interface 13 so as to output the result estimated in the estimation function 153 or the activity of the kinase to be evaluated based on the result. By the output control function 154, the amount of phosphorylated amino acid generated in the substrate protein may be output according to the setting related to the distribution of the immobilization density of the substrate protein received in the reception function 152.

When the distribution of the immobilization density of the substrate protein for each spot is set, the output control function 154 can be performed by, for example, outputting the estimation result of the distribution of the amount of phosphorylated amino acid in each pixel within each spot, outputting the estimation result of the amount of phosphorylated amino acid in the entire spot, or the like. When the distribution is based on the probability distribution, the output control function 154 can be performed by outputting the expected value of the amount of phosphorylated amino acid in the entire spot and the estimation result of the variation thereof, or the like.

When the distribution range of the immobilization density of the substrate protein is set, the output control function 154 can be performed, for example, by outputting an estimation result as the two-dimensional graph of the amount of phosphorylated amino acid in the range. In this case, when the setting includes a range exceeding the range of the immobilization density of the substrate protein present on the substrate actually used for measurement in the kinase activity evaluation apparatus, the output control function 154 can output the estimation result as a fitting curve estimated based on the fitting formula. In addition, when fitting is performed using a parameter that characterizes the fitting formula, for example, the Michaelis-Menten formula, the output control function 154 can output an affinity constant (Km) and a maximum reaction rate (Vmax).

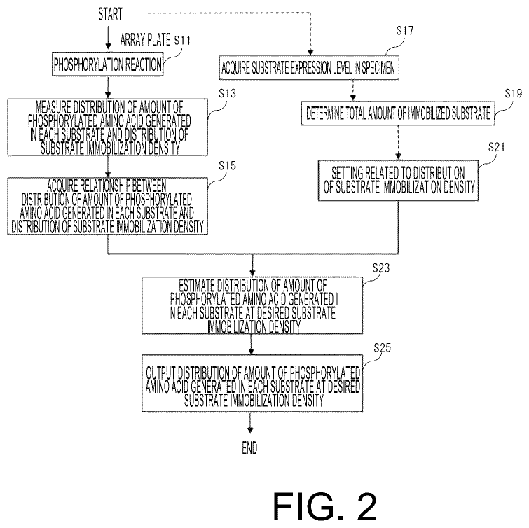

FIG. 2 is a diagram illustrating an example of the flowchart of kinase activity evaluation performed using the kinase activity evaluation apparatus 10 according to the first embodiment. In the following description, a case of outputting the distribution of the amount of phosphorylated amino acid generated in three types of substrate proteins: a substrate protein A, a substrate protein B, and a substrate protein C will be described.

As shown in FIG. 2, first, the kinase activity evaluation apparatus 10 waits until a phosphorylation reaction between kinase and a substrate (protein) is performed by a user on an array plate that is the substrate 100 (step S11). That is, the user brings the specimen into contact with the spot on the carrier to execute the phosphorylation reaction between the kinase contained in the specimen and the substrate protein contained in the spot. Thereby, a phosphorylated substrate protein is obtained.

Next, as shown in FIG. 2, the measurement function 151 in the processing circuit 15 of the kinase activity evaluation apparatus 10 measures the distribution of the amount of phosphorylated amino acid generated in each substrate protein and the distribution of the substrate protein immobilization density (step S13). Specifically, the measurement function 151 measures the distribution of the amount of phosphorylated amino acid generated in the phosphorylated substrate protein and the distribution of the substrate protein immobilization density in the array plate on which the phosphorylation reaction has been executed in step S11, using the confocal optical unit which is the optical system 20. More specifically, the measurement function 151 acquires fluorescence images indicating the amount of immobilization of the substrate protein and the amount of phosphorylated amino acid using a confocal imaging device CID, thereby measuring the distribution of the amount of immobilized substrate protein and the amount of phosphorylated amino acid per pixel within the spot for each substrate protein type.

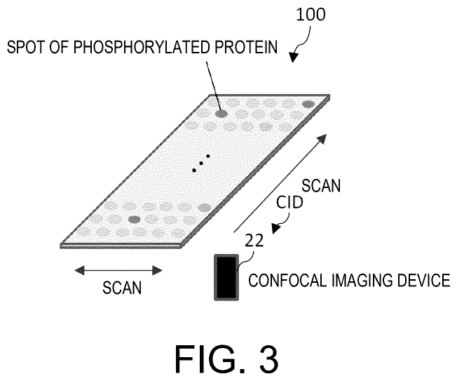

FIG. 3 is a diagram illustrating an example of a method for measuring the distribution of the amount of phosphorylated amino acid generated in a substrate protein and the distribution of a substrate protein immobilization density. As illustrated in FIG. 3, the measurement function 151 causes the confocal imaging device to scan to image the substrate 100 having a spot of phosphorylated protein, thereby measuring the distribution of the amount of phosphorylated amino acid generated in the substrate protein and the distribution of the substrate protein immobilization density.

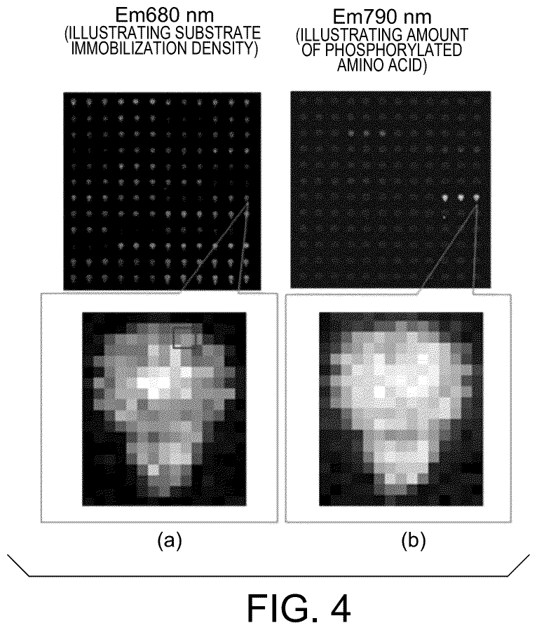

FIG. 4 is a diagram illustrating an example of a measurement result in the first embodiment. As illustrated in FIG. 4, the measurement function 151 acquires a fluorescence image as a measurement result. FIG. 4(a) is the measurement result of the amount of immobilization of the substrate protein (Em680), and FIG. 4(b) is the measurement result of the amount of phosphorylated amino acid (Em790). The upper part of FIG. 4 is a fluorescence image obtained by imaging the substrate 100, and the lower part of FIG. 4 is an enlarged image of a fluorescence image of a spot among a plurality of spots on the substrate 100.

Next, as illustrated in FIG. 2, the measurement function 151 acquires the relationship between the distribution of the amount of phosphorylated amino acid generated in the phosphorylated substrate protein and the distribution of the immobilization density of the substrate protein (step S15). Specifically, the measurement function 151 acquires the relationship between the distribution of the amount of phosphorylated amino acid generated in the phosphorylated substrate protein and the distribution of the immobilization density of the substrate protein, based on the measurement result by the measurement function 151 in step S13. More specifically, the measurement function 151 generates two-dimensional distribution for each substrate protein based on the variation in the substrate protein immobilization density formed within a spot, thereby acquiring the relationship between the distribution of the amount of phosphorylated amino acid for each substrate protein and the distribution of the substrate protein immobilization density.

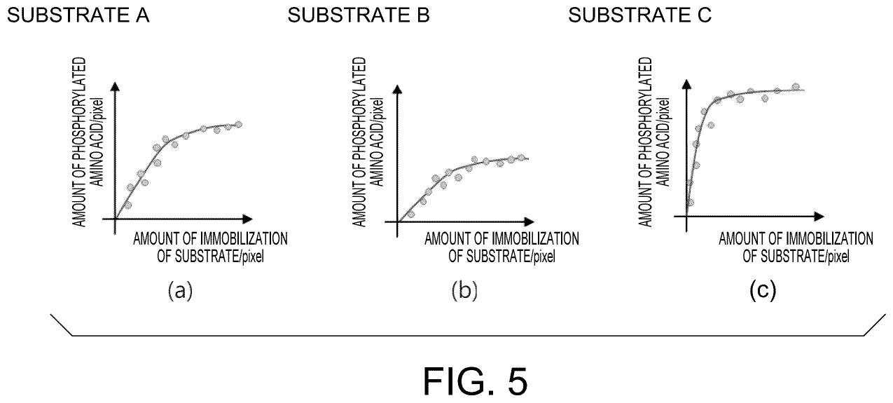

FIG. 5 is a diagram illustrating an example of the relationship between the distribution of the amount of phosphorylated amino acid for each substrate protein and the distribution of the immobilization density of the substrate protein according to the first embodiment. As illustrated in FIG. 5, the measurement function 151 generates two-dimensional distribution for each substrate protein based on the variation in the substrate protein immobilization density formed within a spot as the relationship between the distribution of the amount of phosphorylated amino acid for each substrate protein and the distribution of the substrate protein immobilization density. FIG. 5(a) is two-dimensional distribution of the substrate protein A. FIG. 5(b) is two-dimensional distribution of the substrate protein B. FIG. 5(c) is two-dimensional distribution of the substrate protein C.

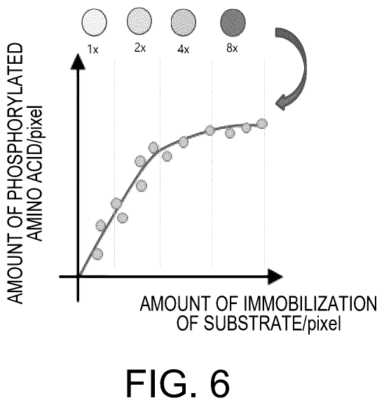

FIG. 6 is a diagram illustrating an example of the relationship between the distribution of the amount of phosphorylated amino acid for each substrate protein and the distribution of the immobilization density of the substrate protein according to the first embodiment. As illustrated in FIG. 6, when the variation in the immobilization density of the substrate protein in each pixel within one spot is produced using a plurality of spots (for example, a spot series prepared using a substrate protein solution in which the substrate protein concentration is stepwisely increased to 1 time, 2 times, 4 times, 8 times, or the like) prepared in advance so that the immobilization density of the substrate protein is different, the measurement function 151 uses the variation in the substrate protein immobilization density within each spot to acquire the relationship between the distribution of the amount of phosphorylated amino acid and the distribution of the immobilization density of the substrate protein.

Next, as illustrated in FIG. 2, the measurement function 151 acquires a substrate protein expression level in a specimen (step S17). Specifically, the measurement function 151 acquires the substrate protein expression level in the specimen measured using a known method such as ELISA (Enzyme-Linked Immuno Sorbent Assay) or LC-MS (Liquid Chromatography-Mass Spectrometer).

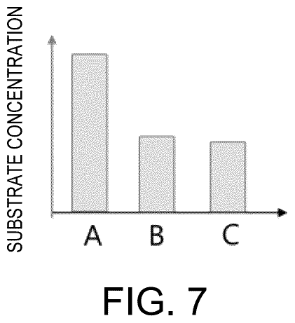

FIG. 7 is a diagram illustrating an example of a substrate protein expression level in a specimen. In the example illustrated in FIG. 7, the substrate protein concentrations of the substrate protein A, the substrate protein B, and the substrate protein C are acquired as the substrate protein expression levels of the substrate protein A, the substrate protein B, and the substrate protein C in the specimens. In the example illustrated in FIG. 7, regarding the substrate protein concentrations of the substrate protein A, the substrate protein B, and the substrate protein C, the substrate protein A shows the highest value, and the substrate protein C shows the lowest value.

Next, as illustrated in FIG. 2, the measurement function 151 determines the total amount of the immobilized substrate protein (step S19). Specifically, the measurement function 151 determines the total amount of the immobilized substrate protein so that the ratio of the respective substrate protein concentrations in the specimen acquired in step S17 coincides with the ratio of the total amount of immobilization per spot.

Next, as illustrated in FIG. 2, the reception function 152 accepts a setting related to the distribution of the substrate protein immobilization density (step S21). Specifically, the reception function 152 receives an input operation related to setting of a parameter related to a spot from the user via the input interface 12, thereby receiving a setting related to the distribution of the substrate protein immobilization density. More specifically, the reception function 152 accepts, via the input interface 12, an input operation on a template for setting a parameter from the user, thereby accepting a setting related to the distribution of the substrate protein immobilization density. In the present embodiment, when the reception function 152 accepts the setting related to the distribution of the substrate protein immobilization density, the distribution of the substrate protein immobilization density is set based on the total amount of the immobilized substrate protein determined in step S19 and the setting related to the distribution of the substrate protein immobilization density.

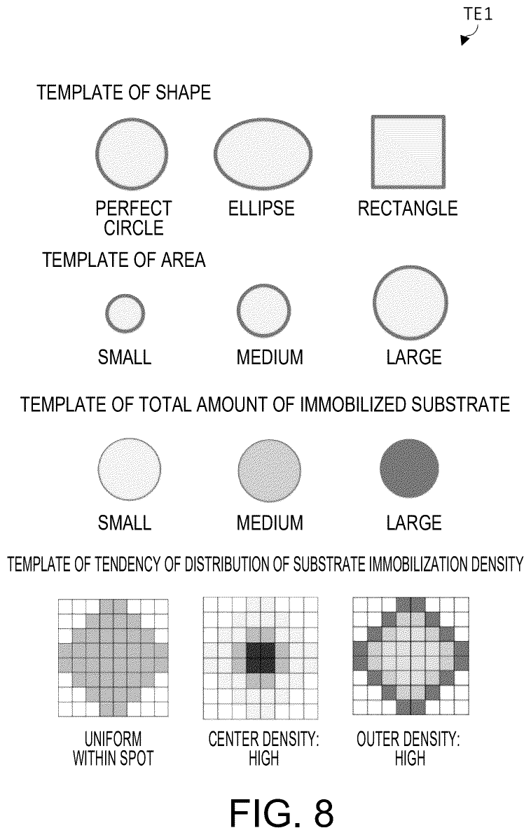

FIG. 8 is a diagram illustrating an example of a template for setting a parameter. In the example illustrated in FIG. 8, a template TE1 includes a template of a shape, a template of an area, a template of the total amount of the immobilized substrate protein, and a template of the tendency of the distribution of the substrate protein immobilization density. In the example illustrated in FIG. 8, “perfect circle”, “ellipse”, and “rectangle” can be set in the template of the shape. In the example illustrated in FIG. 8, “small”, “medium”, and “large” can be set in the template of the area. In the example illustrated in FIG. 8, “small”, “medium”, and “large” can be set in the template of the total amount of immobilization. Furthermore, in the example illustrated in FIG. 8, in the template of the tendency of the distribution of the immobilization density, “uniform within spot”, “center density: high”, and “outer density: high” can be set.

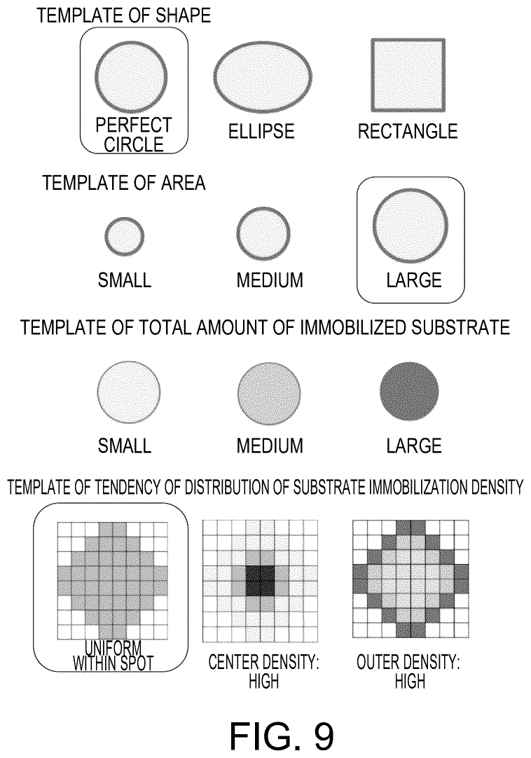

FIG. 9 is a diagram illustrating an example of a parameter setting result according to the first embodiment. In the example illustrated in FIG. 9, the reception function 152 receives, from the user, a setting that the template of the shape is a perfect circle, the template of the area is large, and the template of the tendency of the immobilization density distribution is uniform within spot. In the example illustrated in FIG. 9, the total amount of the immobilized substrate protein is not set using a template of the total amount of the immobilized substrate protein. This is because the total amount of the immobilized substrate protein in step S19 was determined from the expression level of the substrate protein in the specimen acquired in step S17.

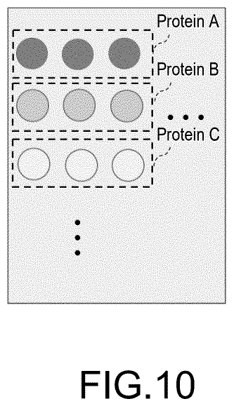

FIG. 10 is a diagram illustrating an example of the setting of the distribution of a substrate protein immobilization density in the first embodiment. As illustrated in FIG. 10, the total amount of immobilization and the immobilization density distribution of each of the substrate protein A, the substrate protein B, and the substrate protein C are set. In the example illustrated in FIG. 10, in the substrate protein concentration indicating the substrate protein expression level in the specimen in FIG. 7, the substrate protein A is high, the substrate protein B is medium, and the substrate protein C is low. Thus, the substrate protein A has a color corresponding to “large” in the template, the substrate protein B has a color corresponding to “medium” in the template, and the substrate protein C has a color corresponding to “small” in the template.

Next, as illustrated in FIG. 2, the estimation function 153 estimates the distribution of the amount of phosphorylated amino acid generated in each substrate protein at a desired substrate protein immobilization density based on the acquired relationship between the distribution of the amount of phosphorylated amino acid generated in the phosphorylated substrate protein and the distribution of the substrate protein immobilization density and the setting related to the distribution of the substrate protein immobilization density (step S23). Specifically, the estimation function 153 estimates the distribution of the amount of phosphorylated amino acid generated in each substrate protein at a desired substrate protein immobilization density according to the distribution of the substrate protein immobilization density set in step S21 based on the acquired relationship between the distribution of the amount of phosphorylated amino acid generated in the phosphorylated substrate protein and the distribution of the substrate protein immobilization density.

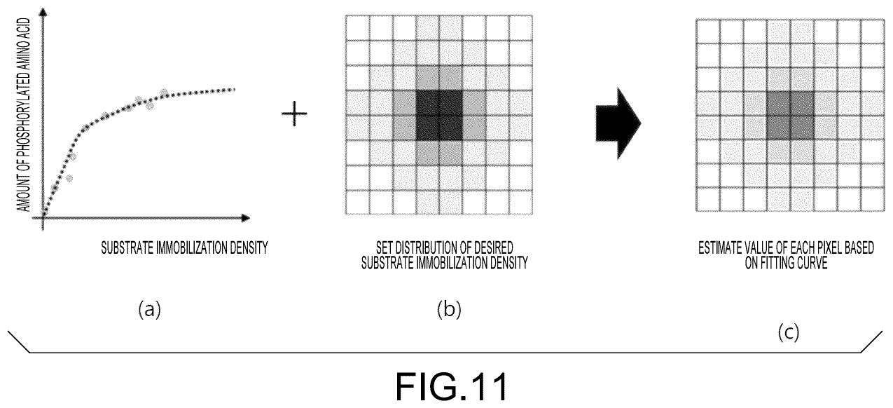

FIG. 11 is a diagram illustrating an example of an estimation result according to the first embodiment. In the example illustrated in FIG. 11, when “center density: high” is set as the distribution of the substrate protein immobilization density as illustrated in FIG. 11(b), and the phosphorylated amino acid density with respect to the substrate protein immobilization density has a constant value as illustrated in FIG. 11(a), the estimation function 153 estimates each pixel value of the distribution of the amount of phosphorylated amino acid generated in each substrate protein at the desired substrate protein immobilization density by determining each pixel value of the distribution of the amount of phosphorylated amino acid generated in each substrate protein at the substrate protein immobilization density according to “center density: high” set as the distribution of the substrate protein immobilization density illustrated in FIG. 11(b) based on the fitting curve of the distribution of the phosphorylated amino acid density with respect to the substrate protein immobilization density illustrated in FIG. 11(a). As a result, the estimation function 153 acquires an estimation result illustrated in FIG. 11(c).

Next, as illustrated in FIG. 2, the output control function 154 outputs the distribution of the amount of phosphorylated amino acid generated in each substrate protein at the estimated desired substrate protein immobilization density (step S25). Specifically, the output control function 154 causes the output interface 13 to output the distribution of the amount of phosphorylated amino acid generated in each substrate protein at the desired substrate protein immobilization density estimated in step S23 according to a method for specifying the substrate protein immobilization density. More specifically, the output control function 154 causes the output interface 13 to output the estimation result of the distribution of the amount of phosphorylated amino acid. As described above, the kinase activity is evaluated in the kinase activity evaluation apparatus 10.

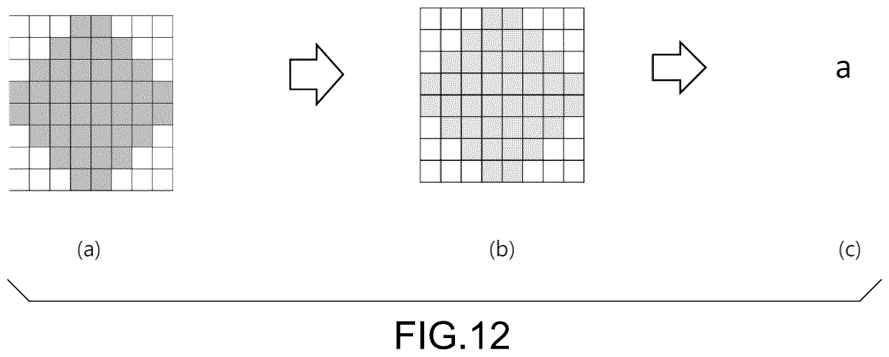

FIG. 12 is a diagram illustrating an example of a method for outputting an estimation result in the first embodiment. When “uniform within spot” is set as the distribution of the substrate protein immobilization density in pixel unit as illustrated in FIG. 12(a), the output control function 154 may output the distribution of the amount of phosphorylated amino acid in each pixel as illustrated in FIG. 12(b), or may output the amount of amino acid in the entire spot as illustrated in FIG. 12(c).

Then, the processing of step S25 is executed, whereby the kinase activity evaluation is terminated.

As described above, the kinase activity evaluation apparatus 10 according to the first embodiment measures the distribution of the immobilization density of the substrate protein and the distribution of the amount of phosphorylated amino acid generated in the substrate protein, in the spot containing the substrate protein on the substrate used for measurement, accepts the setting related to the distribution of the immobilization density of the substrate protein, and estimates the distribution of the amount of phosphorylated amino acid generated in the substrate protein in the distribution of the immobilization density of the substrate protein determined according to the accepted setting, based on the distribution of the immobilization density of the substrate protein and the distribution of the amount of phosphorylated amino acid generated in the substrate protein. Therefore, the user can easily evaluate the kinase activity.

### First Modification

In the kinase activity evaluation apparatus 10 according to the first embodiment described above, the measurement function 151 acquires the substrate protein expression level in the specimen according to measurement using the known method, but it is also possible to acquire a substrate protein expression level in a specimen similar to the specimen as the substrate protein expression level in the specimen. Hereinafter, a case where this modification is applied to the first embodiment will be described as a first modification, and portions different from those of the first embodiment will be described.

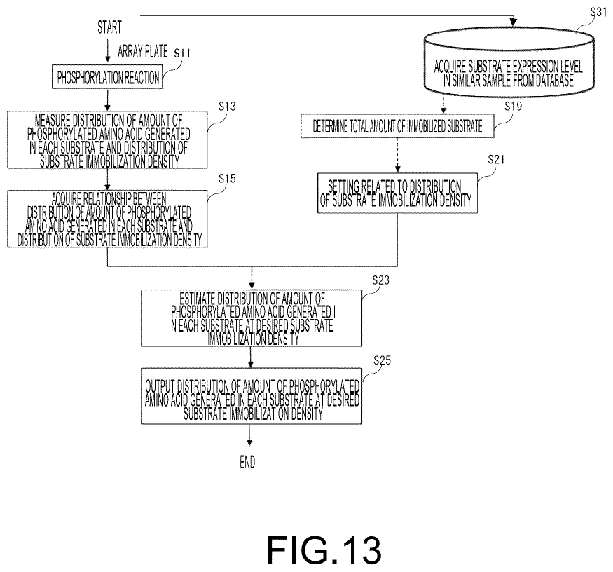

FIG. 13 is a diagram illustrating an example of the flow of kinase activity evaluation performed using a kinase activity evaluation apparatus 10 according to the first modification, and is a diagram corresponding to FIG. 2. Note that the processing in steps S11 to S15 is the same as that in FIG. 2, and thus the description thereof will be omitted.

Next, as illustrated in FIG. 13, the measurement function 151 acquires a substrate protein expression level in a similar sample (specimen) present in database (step S31). Specifically, the measurement function 151 acquires a substrate protein expression level in a specimen similar to the specimen from database included in a storage circuit 14 as the substrate protein expression level in the specimen. Here, the specimen similar to the specimen is the same kind of cell line, a tissue specimen in the same kind of disease patient, or the like. Note that the processing in steps S19 to S25 after step S31 is the same as that in FIG. 2, and thus the description thereof will be omitted. The processing of step S25 is executed, whereby the kinase activity evaluation according to the first modification is terminated.

As described above, also in the kinase activity evaluation apparatus 10 according to the first modification, a user can easily evaluate the kinase activity as in the first embodiment described above.

### Second Modification

In the kinase activity evaluation apparatus 10 according to the first embodiment described above, the distribution of the substrate protein immobilization density is set based on the total amount of the immobilized substrate protein and the setting related to the distribution of the substrate protein immobilization density. However, the distribution of the substrate protein immobilization density can also be set based on the setting related to the distribution of the substrate protein immobilization density. Hereinafter, a case where this modification is applied to the first embodiment will be described as a second modification, and portions different from those of the first embodiment will be described.

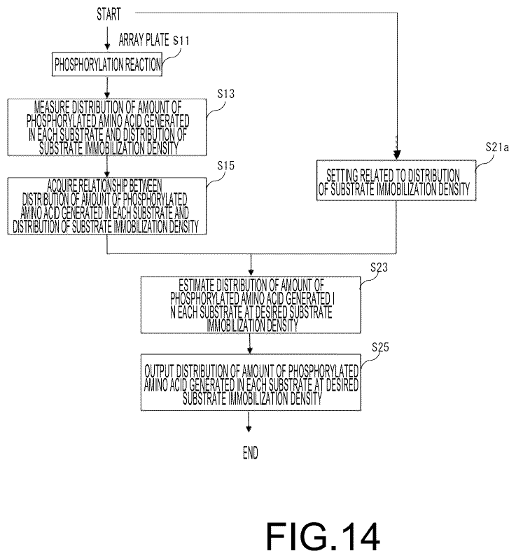

FIG. 14 is a diagram illustrating an example of the flow of kinase activity evaluation performed using a kinase activity evaluation apparatus 10 according to the second modification, and is a diagram corresponding to FIG. 2. Note that the processing in steps S11 to S15 is the same as that in FIG. 2, and thus the description thereof will be omitted.

Next, as illustrated in FIG. 14, a reception function 152 in a processing circuit 15 of the kinase activity evaluation apparatus 10 accepts a setting related to the distribution of the substrate protein immobilization density (step S21a). More specifically, the reception function 152 accepts, via an input interface 12, an input operation on a template for setting a parameter from a user, thereby accepting a setting related to the distribution of the substrate protein immobilization density. In the present modification, when the reception function 152 accepts the setting related to the distribution of the substrate protein immobilization density, the distribution of the substrate protein immobilization density is set based on the setting related to the distribution of the substrate protein immobilization density.

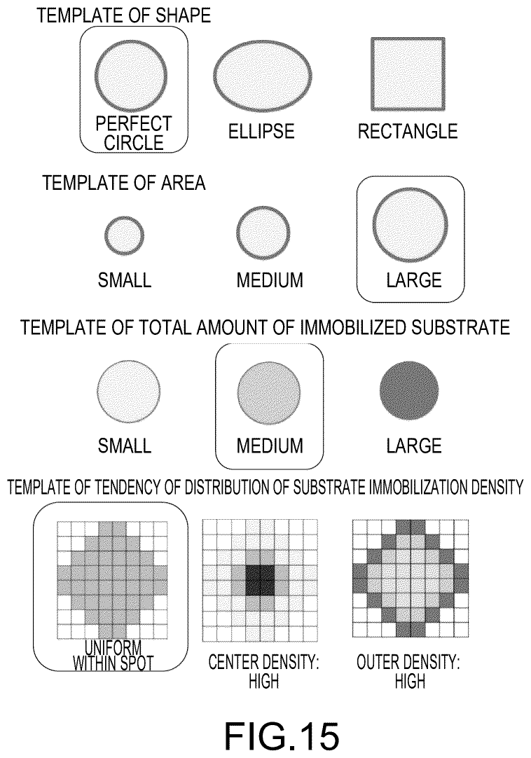

FIG. 15 is a diagram illustrating an example of a parameter setting result in the second modification. In the example illustrated in FIG. 15, the reception function 152 accepts, from the user, a setting that the template of a shape is a perfect circle, the template of an area is large, the template of the total amount of the immobilized substrate protein is medium, and the template of the tendency of the immobilization density distribution is uniform within spot.

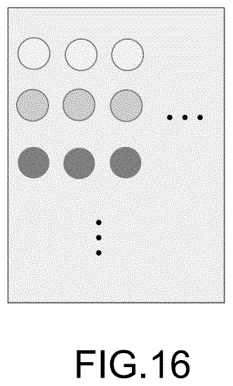

FIG. 16 is a diagram illustrating an example of the setting of the distribution of the substrate protein immobilization density in the second modification. As illustrated in FIG. 16, the total amount of immobilization and the immobilization density distribution of each of a substrate protein A, a substrate protein B, and a substrate protein C are set. In the example illustrated in FIG. 10, the total amount of immobilization of the substrate protein A has a color corresponding to “small” in the template since “small” is selected in the template of the total amount of the immobilized substrate protein in the template. The total amount of immobilization of the substrate protein B has a color corresponding to “medium” in the template since “medium” is selected in the template of the total amount of the immobilized substrate protein in the template. The total amount of immobilization of the substrate protein C has a color corresponding to “large” in the template since “large” is selected in the template of the total amount of the immobilized substrate protein in the template. Note that the processing in step S23 or step S25 after step S21 is the same as that in FIG. 2, and thus the description thereof will be omitted. The processing of step S25 is executed, whereby the kinase activity evaluation according to the second modification is terminated.

As described above, the kinase activity evaluation apparatus 10 according to the second modification receives the setting related to the substrate protein immobilization density from the user without acquiring a substrate protein expression level in a patient's specimen, thereby setting the distribution of the substrate protein immobilization density. Therefore, the distribution of the substrate protein immobilization density can be set more easily than in the first embodiment.

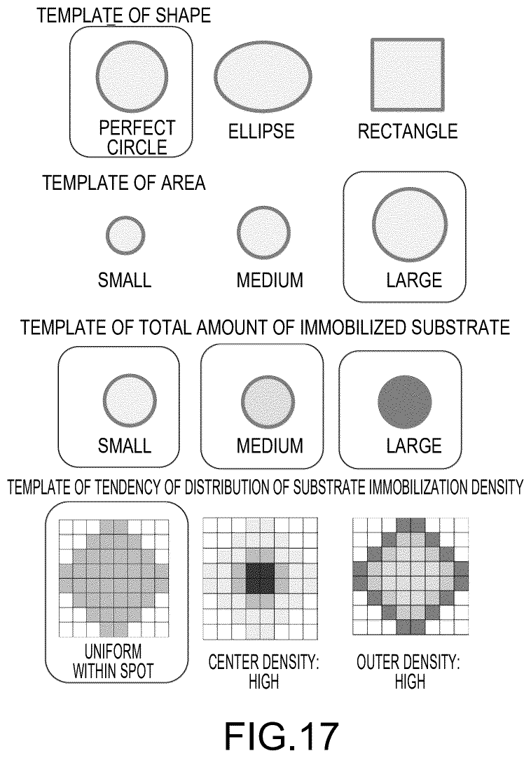

Note that, in the kinase activity evaluation apparatus 10 according to the second modification described above, when the parameter is selected using the template for the same substrate protein species, a plurality of options may be selected from a plurality of options under one condition. FIG. 17 is a diagram illustrating another example of a parameter setting result in the second modification. In the example illustrated in FIG. 17, in “total amount of immobilization_ template” in the template, all of “small”, “medium”, and “large” are selected by the user.

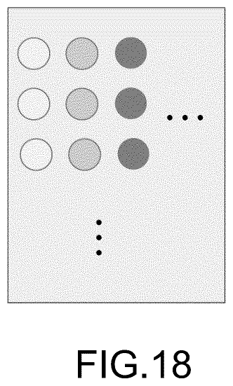

FIG. 18 is a diagram illustrating another example of the setting of the distribution of the substrate protein immobilization density in the second modification, and is a diagram corresponding to FIG. 16. As illustrated in FIG. 18, the total amount of immobilization and the immobilization density distribution of each of the substrate protein A, the substrate protein B, and the substrate protein C are set. In the example illustrated in FIG. 18, regarding the total amount of immobilization of the substrate protein A, all of “small”, “medium”, and “large” are selected in the template of the total amount of the immobilized substrate protein in the template, and thus each spot of the substrate protein A includes a color corresponding to each of “small”, “medium”, and “large”. Also, in the substrate protein B and the substrate protein C, similarly to the substrate protein A, all of “small”, “medium”, and “large” are selected in the template of the total amount of the immobilized substrate protein in the template, and thus each spot of the substrate protein B and the substrate protein C includes a color corresponding to each of “small”, “medium”, and “large”.

As described above, even when the plurality of options is selected from the plurality of options of one condition at the time of selecting the parameter using the template for the same substrate protein species, the immobilization density corresponding to the plurality of options can be set in a substrate protein.

In the kinase activity evaluation apparatus 10 according to the second modification described above, some or all of the parameters are set using the template, but all of the parameters may be arbitrarily set by the user. Specifically, the user may arbitrarily set the shape, the area, the number of spots, the total amount of immobilization within each spot, and the immobilization density distribution within each spot. In order to make the amount of kinase supplied to the spot uniform, it is desirable to make the spot shape and the area constant among the substrate protein species.

### Third Modification

In the kinase activity evaluation apparatus 10 according to the first embodiment described above, the value of the amount of phosphorylated amino acid generated in the substrate protein with respect to the measured value of the immobilization density of the substrate protein can also be expressed as probability distribution. Hereinafter, a case where this modification is applied to the first embodiment will be described as a third modification, and portions different from those of the first embodiment will be described.

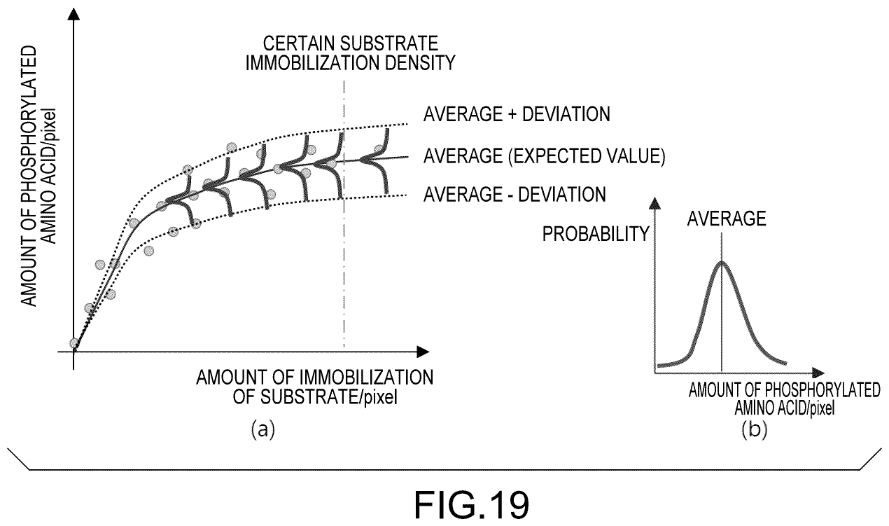

FIG. 19 is a diagram illustrating an example of a relationship between the distribution of the amount of phosphorylated amino acid for each substrate protein and the distribution of the immobilization density of the substrate protein according to a third modification. As illustrated in FIG. 19(a), the value of the amount of phosphorylated amino acid generated in the substrate protein with respect to the measured value of the immobilization density of the substrate protein is expressed as probability distribution. That is, the relationship between the measured distribution of the immobilization density of the substrate protein and the distribution of the amount of phosphorylated amino acid generated in the substrate protein may be determined based on the probability distribution, and the relationship between the measured distribution of the immobilization density for each type of substrate protein and the distribution of the amount of phosphorylated amino acid generated for each type of substrate protein may be determined based on the probability distribution. In this case, the probability distribution of the amount of phosphorylated amino acid generated in the substrate protein with respect to the measured value of the immobilization density of the substrate protein may be expressed in a form having normal distribution illustrated in FIG. 19(b). This probability distribution can be determined from the distribution of the amount of phosphorylated amino acid generated in the same substrate protein with respect to the value of the immobilization density of the substrate protein present on the substrate actually used for measurement in the kinase activity evaluation apparatus 10.

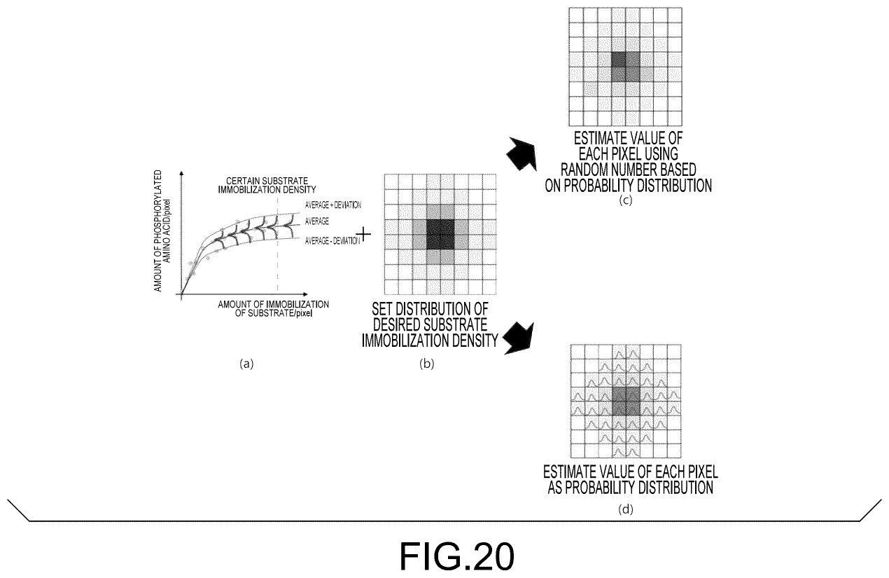

Next, a method for estimating the distribution of the amount of phosphorylated amino acid generated in each substrate protein at a desired substrate protein immobilization density in step S23 of the kinase activity evaluation when the value of the amount of phosphorylated amino acid generated in the substrate protein with respect to the measured value of the immobilization density of the substrate protein is expressed as the probability distribution will be described. FIG. 20 is a diagram illustrating an example of an estimation result in the third modification. In the example illustrated in FIG. 20, when “center density: high” is set as the distribution of the substrate protein immobilization density as illustrated in FIG. 20(b), and the phosphorylated amino acid density with respect to the substrate protein immobilization density has the probability distribution as illustrated in FIG. 20(a), an estimation function 153 estimates each pixel value of the distribution of the amount of phosphorylated amino acid generated in each substrate protein at a desired substrate protein immobilization density by determining the each pixel value of the distribution of the amount of phosphorylated amino acid generated in each substrate protein at the substrate protein immobilization density according to “center density: high” set as the distribution of the substrate protein immobilization density illustrated in FIG. 20(b), based on random numbers based on the probability distribution illustrated in FIG. 20(a). As a result, the estimation function 153 acquires an estimation result illustrated in FIG. 20(c). Alternatively, in the example illustrated in FIG. 20, the estimation function 153 determines, as the probability distribution, each pixel value of the distribution of the amount of phosphorylated amino acid generated in each substrate protein at the substrate protein immobilization density according to “center density: high” set as the distribution of the substrate protein immobilization density illustrated in FIG. 20(b), based on the probability distribution illustrated in FIG. 20(a), thereby estimating each pixel value of the distribution of the amount of phosphorylated amino acid generated in each substrate protein at the desired substrate protein immobilization density. As a result, the estimation function 153 acquires an estimation result illustrated in FIG. 20(d). In the estimation result illustrated in FIG. 20(d), each pixel has the probability distribution for the value of the amount of phosphorylated amino acid.

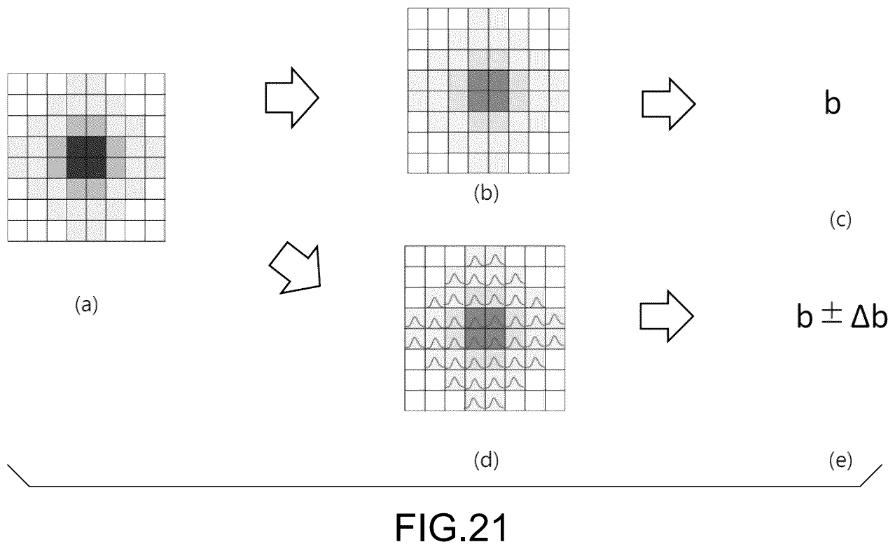

Next, a method for outputting the distribution of the amount of phosphorylated amino acid generated in each substrate protein at the estimated desired substrate protein immobilization density in step S25 of the kinase activity evaluation when the value of the amount of phosphorylated amino acid generated in the substrate protein with respect to the measured value of the immobilization density of the substrate protein is expressed as probability distribution will be described. FIG. 21 is a diagram illustrating an example of a method for outputting an estimation result in the third modification. As illustrated in FIG. 21(a), when “center density: high” is set as the distribution of the substrate protein immobilization density in pixel unit, an output control function 154 may output the distribution of the amount of phosphorylated amino acid in each pixel as illustrated in FIG. 21(b), or may output the amount of amino acid in the entire spot as illustrated in FIG. 21(c). Meanwhile, when “center density: high” is set as the distribution of the substrate protein immobilization density in pixel unit as illustrated in FIG. 21(a), and the value of each pixel in the distribution of the amount of phosphorylated amino acid generated in each substrate protein in the substrate protein immobilization density is determined as the probability distribution as illustrated in FIG. 21(d), the output control function 154 may output the distribution of the amount of phosphorylated amino acid of each pixel in which the value of each pixel is determined as the probability distribution, or may output the expected value of the total amount of phosphorylated amino acid in the entire spot and its variation as illustrated in FIG. 21(e).

As described above, in the kinase activity evaluation apparatus 10 according to the third modification, the value of the amount of phosphorylated amino acid generated in the substrate protein with respect to the measured value of the immobilization density of the substrate protein is expressed as the probability distribution, so that in actual data, the density of phosphorylated amino acid per immobilization density is not constant and may have variation, and thus the magnitude of the variation can be expressed.

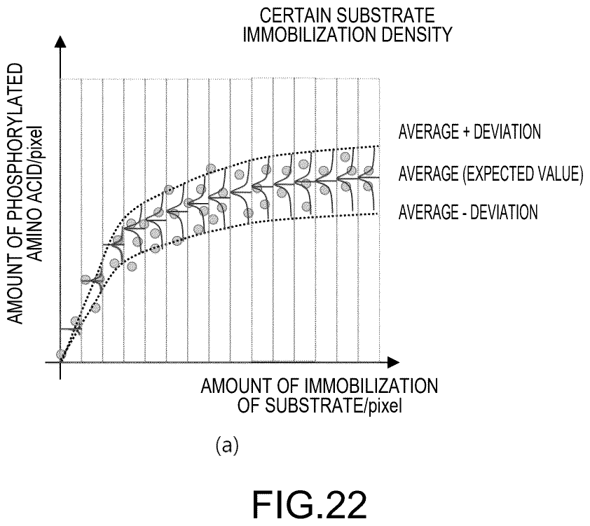

In the kinase activity evaluation apparatus 10 according to the third modification described above, it is also possible to obtain the probability distribution based on the normal distribution by dividing the immobilization density into minute compartments and determining an average value and a standard deviation in each compartment. FIG. 22 is a diagram illustrating another example of a relationship between the distribution of the amount of phosphorylated amino acid for each substrate protein and the distribution of the immobilization density of the substrate protein according to the third modification. Specifically, as illustrated in FIG. 22, a measurement function 151 may acquire probability distribution based on normal distribution by dividing one spot into a plurality of “virtual compartments” that are smaller than one spot and can be set within one spot, and determining an average value and a standard deviation of the amount (this amount may be, for example, an immobilization density) of phosphorylated amino acid generated in the immobilized substrate protein in each “virtual compartment”.

### Fourth Modification

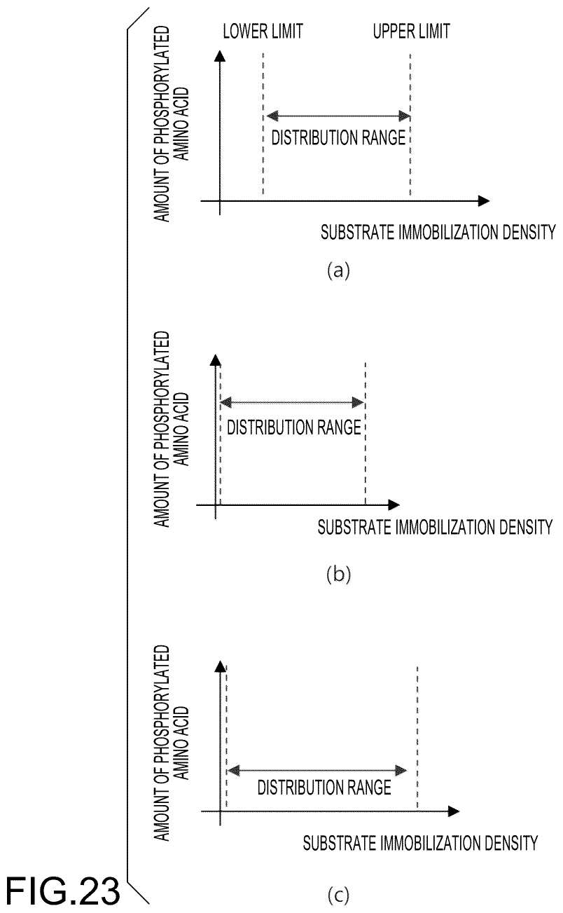

In the kinase activity evaluation apparatus 10 according to the first embodiment described above, the setting of the immobilization density distribution is received for each spot, but the setting of the distribution range of the immobilization density may be received as the setting of the immobilization density distribution. FIG. 23 is a diagram illustrating an example of the setting of the distribution of a substrate protein immobilization density in a fourth modification. FIG. 23(a) illustrates the distribution range of a substrate protein A, FIG. 23(b) illustrates the distribution range of a substrate protein B, and FIG. 23(c) illustrates the distribution range of a substrate protein C. In the example illustrated in FIG. 23, the distribution range of the immobilization density is different in each of the substrate protein A, the substrate protein B, and the substrate protein C.

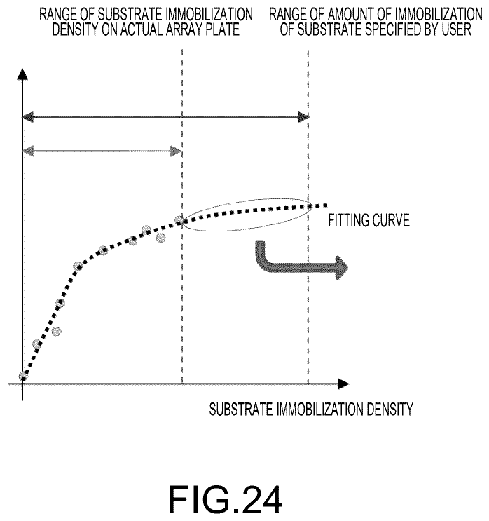

When receiving the setting of the distribution range of the immobilization density as the setting of the immobilization density distribution, an output control function 154 may output the two-dimensional graph of the amount of phosphorylation in the distribution range in which the setting is received as an estimation result. FIG. 24 is a diagram illustrating an example of a method for outputting an estimation result in the fourth modification. As illustrated in FIG. 24, when receiving the setting of the distribution range of the immobilization density as the setting of the immobilization density distribution, the output control function 154 may plot the amount of phosphorylation with respect to the substrate protein immobilization density on an array plate as an estimation result and output a fitting curve thereof. In the example illustrated in FIG. 24, the setting of the distribution range of the immobilization density received as the setting of the immobilization density distribution is set to be wider than the range of the amount of immobilization of the substrate protein on the array plate, and thus the output control function 154 extrapolates the fitting curve estimated based on the fitting formula for a portion wider than the range of the amount of immobilization of the substrate protein on the array plate, and outputs the fitting curve of the range of the amount of immobilization of the substrate protein on the array plate and the extrapolated fitting curve.

When extrapolating the fitting curve estimated based on the fitting formula for a portion wider than the range of the amount of immobilization of the substrate protein on the array plate, the output control function 154 can also additionally output a parameter (Km and Vmax when fitted by the Michaelis-Menten formula) used for fitting the distribution of the immobilization density of the substrate protein and the distribution of the amount of phosphorylated amino acid generated in the substrate protein when presenting the estimation result.

As described above, in the kinase activity evaluation apparatus 10 according to the fourth modification, the setting of the distribution range of the immobilization density is received as the setting of the distribution of the immobilization density, and thus a user can easily set the distribution of the substrate protein immobilization density.

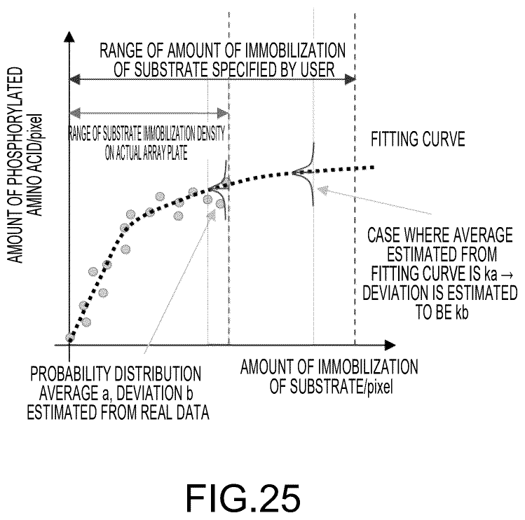

In the kinase activity evaluation apparatus 10 according to the fourth modification, even when the value of the amount of phosphorylated amino acid generated in the substrate protein with respect to the measured value of the immobilization density of the substrate protein is expressed as probability distribution, it is possible to estimate the amount of phosphorylated amino acid generated in the substrate protein by extrapolating the fitting curve for a range exceeding the range of the immobilization density of the substrate protein present on the substrate actually used for measurement in the kinase activity evaluation apparatus 10. FIG. 25 is a diagram illustrating an example of the setting of the distribution of a substrate protein immobilization density in a fourth modification. As illustrated in FIG. 25, even when the value of the amount of phosphorylated amino acid generated in the substrate protein with respect to the measured value of the immobilization density of the substrate protein is expressed as the probability distribution, a measurement function 151 can estimate the probability distribution of the region expanded by the extrapolation based on the probability distribution of the amount of phosphorylated amino acid generated in the substrate protein with respect to the value of the immobilization density of the substrate protein present on the substrate actually used for measurement in the kinase activity evaluation apparatus 10.

### EXAMPLES

The first embodiment described above will be specifically described based on the following examples, but the first embodiment is not limited to these examples. The content is represented in mass% unless otherwise specified.

### Acquisition of Relationship between Distribution of Immobilization Density of Substrate Protein and Distribution of Amount of Phosphorylated Amino Acid Generated in Substrate Protein, Based on Fitting

In the present Example, an example will be described in which a cell extract of a human lung adenocarcinoma cell line PC9 is used as a specimen, various kinase groups contained therein are set as kinases to be measured, a relationship between the distribution of the immobilization density of a substrate protein and the distribution of the amount of phosphorylated amino acids generated in the substrate protein when the immobilized substrate protein MERTK is phosphorylated by the kinase group is acquired, and fitting thereof is performed.

In the present Example, an example will be described in which phosphorylated tyrosine among phosphorylated amino acids is detected, and fitting is performed based on the Michaelis-Menten formula.

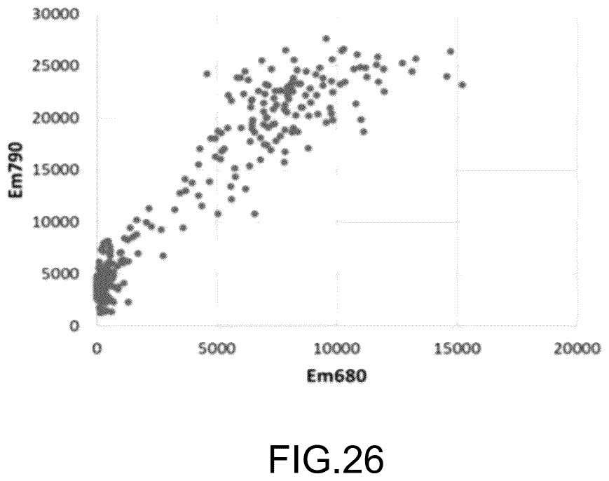

A glutathione-S-transferase (GST) tag-fused MERTK solution as a substrate protein solution was added dropwise to a glutathione (GSH) coated slide glass prepared by a method according to the method described in Non-Patent Document 1 (Tadashi Manabe et al., “IGF2 Autocrine-Mediated IGF1R Activation Is a Clinically Relevant Mechanism of Osimertinib Resistance in Lung Cancer”, Mol Cancer Res. 2020 Apr; 18 (4): 549-559) to obtain an array plate having an immobilized spot of MERTK (diameter: about 100 μm). The cell extract of the human lung adenocarcinoma cell line PC9 was mixed with a kinase reaction solution (25 mM of Tris-HCl, 5 mM of β-glycerophosphate, 0.1 mM of Na₃VO₄, 10 mM of MgCl₂, 1 mM of ATP, and 2 mM of DTT), and the mixture was brought into contact with the MERTK immobilized spot, followed by incubating at 30° C. for 120 minutes to induce a phosphorylation reaction on the spot. Thereafter, a reaction stop solution (50 mM of EDTA, 10 mM of HEPES-NaOH [pH 7.4], 150 mM of NaCl, and 0.05% [v/v] of Tween20) was brought into contact with the immobilized spot, followed by incubating at 30° C. for 5 minutes to stop the phosphorylation reaction. The array plate was washed with TBST, a primary antibody solution (cocktail of mouse anti-phosphotyrosine antibody and rabbit anti GST antibody) was then added, followed by incubating at 30° C. for 1 hour. The array plate was washed with TBST, and a secondary antibody solution (cocktail of Goat anti-Mouse IgG (H+L) Highly Cross-Adsorbed Secondary Antibody, Alexa Fluor 790 (Invitrogen) and Goat anti-Rabbit IgG (H+L) Highly Cross-Adsorbed Secondary Antibody, Alexa Fluor 680 (Invitrogen)) was then added, followed by incubating at 30° C. for 1 hour to label the phosphorylated tyrosine generated in the immobilized spot of MERTK with Alexa Fluor790 and to label the GST tag in MERTK with Alexa Fluor 680. The labeled array plate was measured using a microarray scanner having a confocal optical system to acquire a fluorescence image (FIGS. 3 and 4). For Alexa Fluor 790, measurement was performed using an excitation laser with a wavelength of 790 nm (a fluorescence signal at this time is indicated as Em790nm), and for Alexa Fluor 680, measurement was performed using an excitation laser with a wavelength of 680 nm (a fluorescence signal at this time is indicated as Em680nm). The fluorescence image was obtained with 1 pixel as 10 μm×10 μm, and the distribution of the immobilization density of MERTK and the distribution of the amount of phosphorylated tyrosine generated in MERTK at each point within the spot corresponding to each pixel were acquired from the values of Em680nm and Em790nm at each pixel (FIG. 26). When the distribution of the immobilization density of the substrate protein MERTK and the distribution of the amount of phosphorylated tyrosine are acquired from the fluorescence image, filter processing, particularly a median filter or a mean filter can be applied to the fluorescence image, or processing for reducing noise, particularly moving average processing can also be performed after the acquisition of the distribution.

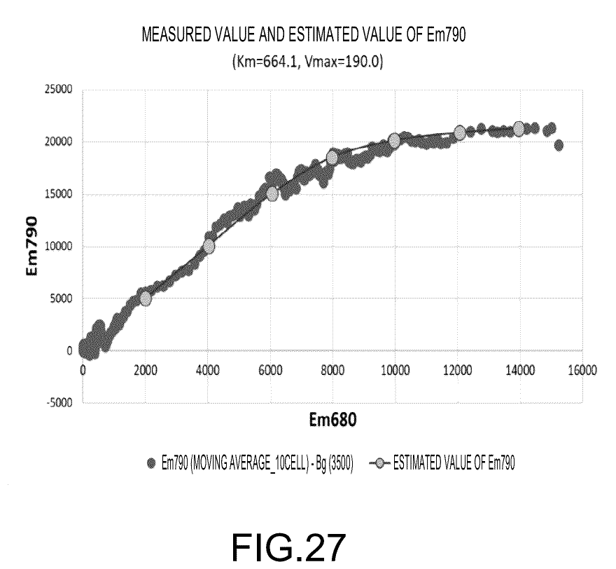

Fitting based on the Michaelis-Menten formula was performed on the distribution of the immobilization density of the acquired substrate protein MERTK and the distribution of the amount of phosphorylated tyrosine. In general, the Michaelis-Menten formula is a formula indicating a relationship between a substrate protein concentration and an enzyme reaction rate. Meanwhile, the distribution of the immobilization density of the substrate protein acquired by the above procedure and the distribution of the amount of phosphorylated tyrosine show the substrate protein density at a certain point within a spot at the start of the reaction (0 min) and the distribution of the integrated amount of phosphorylated tyrosine generated by the end of the reaction (120 min). Therefore, in order to perform fitting by the Michaelis-Menten formula, it is necessary to consider that the unreacted substrate protein density decreases with the progress of a phosphorylation reaction and to determine the integrated amount of phosphorylated tyrosine until the end of the reaction as the time integral of a phosphorylation reaction rate. Based on the above, the fitting using the Michaelis-Menten formula was performed according to the following procedure. When certain Km and Vmax were assumed, the production rate of phosphorylated tyrosine and the amount of decrease in unreacted MERTK after the lapse of unit time in the density of MERTK at the reaction start time point acquired above were determined. Based on the density of unreacted MERTK after reduction, the production rate of phosphorylated tyrosine at the next unit time was determined. By repeating this operation until a phosphorylation reaction time (120 minutes) was reached, the correspondence relationship of the amount of phosphorylated tyrosine with respect to the immobilization density of MERTK was estimated (FIG. 27). Vmax and Km were converged to minimize the error between estimated correspondence relationship and the relationship of the immobilization density of MERTK and the amount of phosphorylated tyrosine actually acquired from the array plate, thereby acquiring Vmax and Km. The amount of phosphorylated amino acid generated in the substrate protein was estimated by extrapolating a fitting curve based on the acquired Vmax and Km also for a range exceeding the range of the immobilization density of the substrate protein present in the actual array plate.

### Acquisition of Relationship between Distribution of Immobilization Density of Substrate Protein and Distribution of Amount of Phosphorylated Amino Acid Generated in Substrate Protein, Based on Probability Distribution

In the distribution of the immobilization density of the substrate protein acquired in the actual array plate and the distribution of the amount of phosphorylated amino acid generated in the substrate protein, even if the immobilization density of the substrate protein is the same, the amount of phosphorylated amino acid is distributed with a certain variation, and thus an example in which this variation is treated as the probability distribution will be described.

The distribution of the immobilization density of the substrate protein (here, MERTK) and the distribution of the amount of phosphorylated amino acid (here, phosphorylated tyrosine) were acquired by the same procedure as that of the acquisition of the relationship between the distribution of the immobilization density of the substrate protein and the distribution of the amount of phosphorylated amino acid generated in the substrate protein, based on fitting. Regarding the distribution, fluorescent luminance Em680 indicating the immobilization density of MERTK was divided into certain minute ranges (range of about 1000 each), and the average value and standard deviation of the amount of phosphorylated tyrosine in each range were obtained. The amount of phosphorylated tyrosine at a point having the immobilization density of MERTK belonging to each range was acquired as the probability distribution of normal distribution having the average value and standard deviation of the amount of phosphorylated tyrosine in the range. When the number of data belonging to each minute range of Em680 was small, as another means for obtaining the probability distribution, the distribution of the immobilization density of the substrate protein and the distribution of the amount of phosphorylated amino acid were obtained by the same procedure as that of the acquisition of the relationship between the distribution of the immobilization density of the substrate protein and the distribution of the amount of phosphorylated amino acid generated in the substrate protein, based on fitting, and then the distribution data was arranged in order of the magnitude of Em680 to acquire the moving average value and the moving standard deviation of Em680nm and Em790nm, thereby acquiring the probability distribution as the normal distribution of the average value and standard deviation of the amount of phosphorylated amino acid with respect to the moving average value of certain substrate protein immobilization density.

In order to acquire the probability distribution of the amount of phosphorylated amino acid with respect to the substrate protein immobilization density distribution for a range exceeding the range of the immobilization density of the substrate protein present in the actual array plate, the results of fitting performed in the same manner as in the acquisition of the relationship between the distribution of the immobilization density of the substrate protein and the distribution of the amount of phosphorylated amino acid generated in the substrate protein, based on fitting were used. That is, when the average value of the amounts of phosphorylated amino acids was a and the standard deviation was b in a certain compartment (a compartment having a high immobilization density when a fitting curve is extrapolated to a region having a high immobilization density) of the immobilization density where data was actually present, and when the fitting value of the amount of phosphorylated amino acid at the substrate protein immobilization density in a range in which the fitting curve was extrapolated beyond the range of the immobilization density of the substrate protein present in the actual array plate was ka, it was considered that the amount of phosphorylated amino acid at the substrate protein immobilization density had normal distribution with an average value of ka and a standard deviation of kb.

### Setting of Distribution of Immobilization Density of Substrate Protein

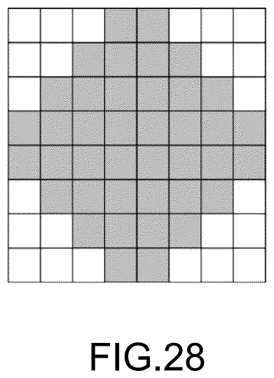

As desired by the user of the apparatus, the distribution of the immobilization density was set for various substrate proteins and input to a reception function 152. As the setting of the distribution of the immobilization density, a parameter such as “area, shape, number of spots, total amount of immobilization within spot, distribution shape of substrate protein immobilization density” as a spot was set, or simply set as the distribution range of the immobilization density (in this case, setting as the spot is not performed). In the case of setting as the spot, the apparatus user arbitrarily sets individual parameters for various substrate proteins one by one or selects the parameter of the spot or the set thereof from a template in order to avoid complicated setting. In the example shown in FIG. 28, the reception function 152 accepts the setting of “average within spot” as the template of the tendency of the distribution of the substrate protein immobilization density desired by the user via the template.

The ratio of the total amount of substrate protein immobilization for each type of substrate protein can be the same as the ratio of the concentration and expression level for each type of substrate protein in the specimen (measurable by a known method such as ELISA or LC-MS). In this case, analysis reflecting the concentration of the substrate protein in the specimen can be performed, and a manner in which a phosphorylation reaction rate by kinase is controlled in a concentration-dependent manner of the substrate protein, such as a case where the concentration of the substrate protein in the specimen is small and the phosphorylation reaction rate by kinase is limited, can be acquired as the estimation result of the estimation of the distribution of the amount of phosphorylated amino acid generated in the substrate protein in the distribution of the immobilization density of the substrate protein determined according to the setting when the amount of phosphorylated amino acid generated in the substrate protein with respect to the immobilization density of the substrate protein described later has a constant value, or the estimation result of the estimation of the distribution of the amount of phosphorylated amino acid generated in the substrate protein in the distribution of the immobilization density of the substrate protein determined according to the setting when the amount of phosphorylated amino acid generated in the substrate protein with respect to the immobilization density of the substrate protein has probability distribution. The concentration and expression level for each type of substrate protein in the specimen can be measured for each specimen each time the kinase activity evaluation is performed. However, when a sample similar to a sample whose expression level of the substrate protein is known is used as the specimen, it is also possible to set the ratio of the total amount of substrate protein immobilization for each type of substrate protein with reference to the expression level of the substrate protein in the known sample. Depending on the purpose of the kinase activity evaluation, in particular, in the case of the purpose of obtaining the maximum value of the kinase activity under the condition that the concentration of the substrate protein is high and the reaction rate is not limited by the concentration of the substrate protein, even if the ratio of the concentration and the expression level for each type of substrate protein in the specimen is known, the ratio of the total amount of substrate protein immobilization for each type of substrate protein to be set does not need to be the same as the ratio in the specimen.

### Estimation of Distribution of Amount of Phosphorylated Amino Acid Generated in Substrate Protein in Distribution of Immobilization Density of Substrate Protein Determined According to Setting

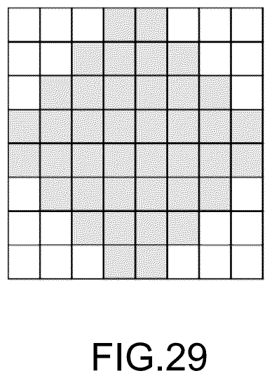

When fitting is performed for the distribution of the immobilization density of the substrate protein acquired in the actual array plate and the distribution of the amount of phosphorylated amino acid generated in the substrate protein by the procedure for acquiring the relationship between the distribution of the immobilization density of the substrate protein and the distribution of the amount of phosphorylated amino acid generated in the substrate protein, based on fitting, the relationship between the distribution of the immobilization density of the substrate protein and the distribution of the amount of phosphorylated amino acid generated in the substrate protein can be determined so that the amount of phosphorylated amino acid generated in the substrate protein with respect to the immobilization density of the substrate protein takes a constant value. In this case, the amount of phosphorylated amino acid generated in the substrate protein in the distribution of the immobilization density of the substrate protein set in a manner desired by the user of the apparatus in the procedure of setting the distribution of the immobilization density of the substrate protein was uniquely estimated from the relationship between the substrate protein immobilization density and the amount of phosphorylated amino acid (FIG. 29).

The estimation result of the amount of phosphorylated amino acid generated in the substrate protein in the distribution of the immobilization density of the substrate protein determined according to the setting was presented according to the method for setting the substrate protein immobilization density. When the substrate protein immobilization density distribution as the spot was set, the estimation result of the distribution of the amount of phosphorylated amino acid at each point of the spot was presented as an image or numerical data, and the amount of phosphorylated amino acid in the entire spot was presented as numerical data (FIG. 12). When the distribution range of the immobilization density of the substrate protein was set (FIG. 23), a graph of the distribution of the amount of phosphorylated amino acid in the distribution range was presented. Note that, regardless of the method for specifying the substrate protein immobilization density distribution, when presenting the estimation result, it is also possible to additionally present parameters (Km and Vmax when fitted by the Michaelis-Menten formula) used for fitting the distribution of the immobilization density of the substrate protein and the distribution of the amount of phosphorylated amino acid generated in the substrate protein.

Estimation of Distribution of Amount of Phosphorylated Amino Acid Generated in Substrate Protein in Distribution of Immobilization Density of Substrate Protein Determined According to Setting When Amount of Phosphorylated Amino Acid Generated in Substrate Protein with Respect to Immobilization Density of Substrate Protein Has Probability Distribution

When fitting was performed for the distribution of the immobilization density of the substrate protein acquired in the actual array plate and the distribution of the amount of phosphorylated amino acid generated in the substrate protein by the procedure of acquiring the relationship between the distribution of the immobilization density of the substrate protein and the distribution of the amount of phosphorylated amino acid generated in the substrate protein, based on the probability distribution, the relationship of the probability distribution of the amount of phosphorylated amino acid generated in the substrate protein with respect to the immobilization density of the substrate protein was acquired. In this case, the amount of phosphorylated amino acid generated in the substrate protein in the distribution of the immobilization density of the substrate protein set in the manner desired by the user of the apparatus in the procedure of setting the distribution of the immobilization density of the substrate protein was also estimated in the manner of the probability distribution in light of the above relationship.

The estimation result of the amount of phosphorylated amino acid generated in the substrate protein in the distribution of the immobilization density of the substrate protein determined according to the setting was presented according to the method for setting the substrate protein immobilization density. When the substrate protein immobilization density distribution as the spot was set, the estimation result of the probability distribution of the amount of phosphorylated amino acid at each point of the spot was presented as an image or numerical data, and the expected value of the amount of phosphorylated amino acid in the entire spot and its deviation were presented as numerical data (FIG. 21). When the distribution range of the immobilization density of the substrate protein was set, the estimation result was presented as the transition of the average value and deviation of the amount of phosphorylated amino acid in the distribution range.

At least one of the embodiments described above allows evaluation of kinase activity easily.

While certain embodiments have been described, these embodiments have been presented by way of example only and are not intended to limit the scope of the inventions. The embodiments may be in a variety of other forms. Furthermore, various omissions, substitutions and changes may be made without departing from the spirit of the inventions. The embodiments and their modifications are included in the scope and the subject matter of the invention, and at the same time included in the scope of the claimed inventions and their equivalents.

1. An apparatus for evaluating kinase activity, the apparatus comprising:
processing circuitry configured to
measure distribution of an immobilization density of a substrate protein and distribution of an amount of phosphorylated amino acid generated in the substrate protein, in a spot containing the substrate protein on a substrate used for measurement;
accept a setting related to the distribution of the immobilization density of the substrate protein; and
estimate the distribution of the amount of phosphorylated amino acid generated in the substrate protein in the distribution of the immobilization density of the substrate protein determined according to the received setting, based on the distribution of the immobilization density of the substrate protein and the distribution of the amount of phosphorylated amino acid generated in the substrate protein.

2. The apparatus of claim 1,
wherein the measurement further acquires a relationship between the measured distribution of the immobilization density of the substrate protein and the distribution of the amount of phosphorylated amino acid generated in the substrate protein.

3. The apparatus of claim 1,
wherein the processing circuitry is further configured to acquire a relationship between the measured distribution of the immobilization density of the substrate protein and the distribution of the amount of phosphorylated amino acid generated in the substrate protein.

4. The apparatus of claim 2,
wherein the relationship between the measured distribution of the immobilization density of the substrate protein and the distribution of the amount of phosphorylated amino acid generated in the substrate protein is determined based on fitting.

5. The apparatus of claim 2,
wherein the relationship between the measured distribution of the immobilization density of the substrate protein and the distribution of the amount of phosphorylated amino acid generated in the substrate protein is determined based on probability distribution.

6. The apparatus of claim 1,
wherein the accepted setting related to the distribution of the immobilization density of the substrate protein includes one or more selected from the group consisting of an area and shape of each spot containing the substrate protein and a total amount of the immobilized substrate protein in each spot.

7. The apparatus of claim 6,
wherein a ratio of an amount of another specific substrate protein to an amount of a specific substrate protein in each spot and a ratio of an amount of another specific substrate protein to an amount of a specific substrate protein in a specimen containing kinase to be evaluated are at least partially the same.

8. A non-transitory computer readable storage medium, which stores a program that causes an apparatus for evaluating kinase activity to execute processing comprising:
measuring distribution of an immobilization density of a substrate protein and distribution of an amount of phosphorylated amino acid generated in the substrate protein, in a spot containing the substrate protein on a substrate used for measurement;
accepting a setting related to the distribution of the immobilization density of the substrate protein; and
estimating the distribution of the amount of phosphorylated amino acid generated in the substrate protein in the distribution of the immobilization density of the substrate protein determined according to the received setting, based on the distribution of the immobilization density of the substrate protein and the distribution of the amount of phosphorylated amino acid generated in the substrate protein.

9. The non-transitory computer readable storage medium according to claim 8,
wherein the measuring further comprises acquiring a relationship between the measured distribution of the immobilization density of the substrate protein and the distribution of the amount of phosphorylated amino acid generated in the substrate protein.

10. The non-transitory computer readable storage medium according to claim 8, further comprising:
acquiring a relationship between the measured distribution of the immobilization density of the substrate protein and the distribution of the amount of phosphorylated amino acid generated in the substrate protein.

11. The non-transitory computer readable storage medium according to claim 9,
wherein the relationship between the measured distribution of the immobilization density of the substrate protein and the distribution of the amount of phosphorylated amino acid generated in the substrate protein is determined based on fitting.

12. The non-transitory computer readable storage medium according to claim 9,
wherein the relationship between the measured distribution of the immobilization density of the substrate protein and the distribution of the amount of phosphorylated amino acid generated in the substrate protein is determined based on probability distribution.

13. The non-transitory computer readable storage medium according to claim 8,
wherein the accepted setting related to the distribution of the immobilization density of the substrate protein includes one or more selected from the group consisting of an area and shape of each spot containing the substrate protein and a total amount of the immobilized substrate protein in each spot.

14. The non-transitory computer readable storage medium according to claim 13,
wherein a ratio of an amount of another specific substrate protein to an amount of a specific substrate protein in each spot and a ratio of an amount of another specific substrate protein to an amount of a specific substrate protein in a specimen containing kinase to be evaluated are at least partially the same.
